SEQUENCE LISTING (110 sequences)

SEQ ID NO: 1 - protein, 1988 aa, Homo sapiens
  REGION 1..1988: note = UniProt. No. Q15858-1
  source 1..1988: note = GenBank accession number ENSG00000169432
MAMLPPPGPQSFVHFTKQSLALIEQRIAERKSKEPKEEKKDDDEEAPKPSSDLEAGKQLPFIYGDIPPGMVSEPLEDLDPYYADKKTFIVLNKGKTIFRFNATPALYMLSPFSPLRRISIKILVHSLFSMLIMCTILTNCIFMTMNNPPDWTKNVEYTFTGIYTFESLVKILARGFCVGEFTFLRDPWNWLDFVVIVFAYLTEFVNLGNVSALRTFRVLRALKTISVIPGLKTIVGALIQSVKKLSDVMILTVFCLSVFALIGLQLFMGNLKHKCFRNSLENNETLESIMNTLESEEDFRKYFYYLEGSKDALLCGFSTDSGQCPEGYTCVKIGRNPDYGYTSFDTFSWAFLALFRLMTQDYWENLYQQTLRAAGKTYMIFFVVVIFLGSFYLINLILAVVAMAYEEQNQANIEEAKQKELEFQQMLDRLKKEQEEAEAIAAAAAEYTSIRRSRIMGLSESSSETSKLSSKSAKERRNRRKKKNQKKLSSGEEKGDAEKLSKSESEDSIRRKSFHLGVEGHRRAHEKRLSTPNQSPLSIRGSLFSARRSSRTSLFSFKGRGRDIGSETEFADDEHSIFGDNESRRGSLFVPHRPQERRSSNISQASRSPPMLPVNGKMHSAVDCNGVVSLVDGRSALMLPNGQLLPEVIIDKATSDDSGTTNQIHKKRRCSSYLLSEDMLNDPNLRQRAMSRASILTNTVEELEESRQKCPPWWYRFAHKFLIWNCSPYWIKFKKCIYFIVMDPFVDLAITICIVLNTLFMAMEHHPMTEEFKNVLAIGNLVFTGIFAAEMVLKLIAMDPYEYFQVGWNIFDSLIVTLSLVELFLADVEGLSVLRSFRLLRVFKLAKSWPTLNMLIKIIGNSVGALGNLTLVLAIIVFIFAVVGMQLFGKSYKECVCKINDDCTLPRWHMNDFFHSFLIVFRVLCGEWIETMWDCMEVAGQAMCLIVYMMVMVIGNLVVLNLFLALLLSSFSSDNLTAIEEDPDANNLQIAVTRIKKGINYVKQTLREFILKAFSKKPKISREIRQAEDLNTKKENYISNHTLAEMSKGHNFLKEKDKISGFGSSVDKHLMEDSDGQSFIHNPSLTVTVPIAPGESDLENMNAEELSSDSDSEYSKVRLNRSSSSECSTVDNPLPGEGEEAEAEPMNSDEPEACFTDGCVWRFSCCQVNIESGKGKIWWNIRKTCYKIVEHSWFESFIVLMILLSSGALAFEDIYIERKKTIKIILEYADKIFTYIFILEMLLKWIAYGYKTYFTNAWCWLDFLIVDVSLVTLVANTLGYSDLGPIKSLRTLRALRPLRALSRFEGMRVVVNALIGAIPSIMNVLLVCLIFWLIFSIMGVNLFAGKFYECINTTDGSRFPASQVPNRSECFALMNVSQNVRWKNLKVNFDNVGLGYLSLLQVATFKGWTIIMYAAVDSVNVDKQPKYEYSLYMYIYFVVFIIFGSFFTLNLFIGVIIDNFNQQKKKLGGQDIFMTEEQKKYYNAMKKLGSKKPQKPIPRPGNKIQGCIFDLVTNQAFDISIMVLICLNMVTMMVEKEGQSQHMTEVLYWINVVFIILFTGECVLKLISLRHYYFTVGWNIFDFVVVIISIVGMFLADLIETYFVSPTLFRVIRLARIGRILRLVKGAKGIRTLLFALMMSLPALFNIGLLLFLVMFIYAIFGMSNFAYVKKEDGINDMFNFETFGNSMICLFQITTSAGWDGLLAPILNSKPPDCDPKKVHPGSSVEGDCGNPSVGIFYFVSYIIISFLVVVNMYIAVILENFSVATEESTEPLSEDDFEMFYEVWEKFDPDATQFIEFSKLSDFAAALDPPLLIAKPNKVQLIAMDLPMVSGDRIHCLDILFAFTKRVLGESGEMDSLRSQMEERFMSANPSKVSYEPITTTLKRKQEDVSATVIQRAYRRYRLRQNVKNISSIYIKDGDRDDDLLNKKDMAFDNVNENSSPEKTDATSSTTSPPSYDSVTKPDKEKYEQDRTEKEDKGKDSKESKK

SEQ ID NO: 2 - protein, 96 aa, Homo sapiens
  REGION 1..96: note = KRAB domain sequence
  source 1..96: note = GenBank No. NM_015394.4
DAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 3 - protein, 5 aa, synthetic construct
  REGION 1..5: note = linker
DGGGS

SEQ ID NO: 4 - protein, 5 aa, synthetic construct
  REGION 1..5: note = linker
TGEKP

SEQ ID NO: 5 - protein, 9 aa, synthetic construct
  REGION 1..9: note = linker
LRQKDGERP

SEQ ID NO: 6 - protein, 4 aa, synthetic construct
  REGION 1..4: note = linker
GGRR

SEQ ID NO: 7 - protein, 8 aa, synthetic construct
  REGION 1..8: note = linker
GGRRGGGS

SEQ ID NO: 8 - protein, 9 aa, synthetic construct
  REGION 1..9: note = linker
LRQRDGERP

SEQ ID NO: 9 - protein, 12 aa, synthetic construct
  REGION 1..12: note = linker
LRQKDGGGSERP

SEQ ID NO: 10 - protein, 16 aa, synthetic construct
  REGION 1..16: note = linker
LRQKDGGGSGGGSERP

SEQ ID NO: 11 - protein, 6 aa, synthetic construct
  REGION 1..6: note = linker
TGSQKP

SEQ ID NO: 12 - skipped

SEQ ID NO: 13 - protein, 10 aa, synthetic construct
  REGION 1..10: note = linker
LRQKDAARGS

SEQ ID NO: 14 - protein, 12 aa, synthetic construct
  REGION 1..12: note = linker
LRQKDAARGSGG

SEQ ID NO: 15 - protein, 5 aa, synthetic construct
  REGION 1..5: note = linker
GGGGS

SEQ ID NO: 16 - DNA, 28 bp, synthetic construct
  misc_feature 1..28: note = DNA-binding recognition helix sequences
  source 1..28: mol_type = other DNA
gtggtggcgacgctgtagcctctgcacc

SEQ ID NO: 17 - DNA, 28 bp, synthetic construct
  misc_feature 1..28: note = DNA-binding recognition helix sequences
  source 1..28: mol_type = other DNA
gtggcgacgctgtagcctctgcaccatg

SEQ ID NO: 18 - DNA, 28 bp, synthetic construct
  misc_feature 1..28: note = DNA-binding recognition helix sequences
  source 1..28: mol_type = other DNA
ttgcaggcggtcgccagcgctccagcgg

SEQ ID NO: 19 - DNA, 28 bp, synthetic construct
  misc_feature 1..28: note = DNA-binding recognition helix sequences
  source 1..28: mol_type = other DNA
agagaggggagaagcttgaccgggtggt

SEQ ID NO: 20 - DNA, 28 bp, synthetic construct
  misc_feature 1..28: note = DNA-binding recognition helix sequences
  source 1..28: mol_type = other DNA
tggccgtggatggcaggtcgtgcaaccc

SEQ ID NO: 21 - DNA, 28 bp, synthetic construct
  misc_feature 1..28: note = DNA-binding recognition helix sequences
  source 1..28: mol_type = other DNA
tatttgtgtttgtgtgctcttaaggggt

SEQ ID NO: 22 - DNA, 28 bp, synthetic construct
  misc_feature 1..28: note = DNA-binding recognition helix sequences
  source 1..28: mol_type = other DNA
gtgcagaggctacagcgtcgccaccacc

SEQ ID NO: 23 - DNA, 28 bp, synthetic construct
  misc_feature 1..28: note = DNA-binding recognition helix sequences
  source 1..28: mol_type = other DNA
agggggttgcacgacctgccatccacgg

SEQ ID NO: 24 - DNA, 28 bp, synthetic construct
  misc_feature 1..28: note = DNA-binding recognition helix sequences
  source 1..28: mol_type = other DNA
cgcatggtgcagaggctacagcgtcgcc

SEQ ID NO: 25 - protein, 7 aa, synthetic construct
  REGION 1..7: note = DNA-Recognition Helix Amino Acid Sequence Within Each Finger
DRSDLSR

SEQ ID NO: 26 - protein, 7 aa, synthetic construct
  REGION 1..7: note = DNA-Recognition Helix Amino Acid Sequence Within Each Finger
QSGSLTR

SEQ ID NO: 27 - protein, 7 aa, synthetic construct
  REGION 1..7: note = DNA-Recognition Helix Amino Acid Sequence Within Each Finger
QSGDLTR

SEQ ID NO: 28 - protein, 7 aa, synthetic construct
  REGION 1..7: note = DNA-Recognition Helix Amino Acid Sequence Within Each Finger
DSSNRAK

SEQ ID NO: 29 - protein, 7 aa, synthetic construct
  REGION 1..7: note = DNA-Recognition Helix Amino Acid Sequence Within Each Finger
DRSHLSR

SEQ ID NO: 30 - protein, 7 aa, synthetic construct
  REGION 1..7: note = DNA-Recognition Helix Amino Acid Sequence Within Each Finger
TSGHLSR

SEQ ID NO: 31 - protein, 7 aa, synthetic construct
  REGION 1..7: note = DNA-Recognition Helix Amino Acid Sequence Within Each Finger
ASKTRTN

SEQ ID NO: 32 - protein, 7 aa, synthetic construct
  REGION 1..7: note = DNA-Recognition Helix Amino Acid Sequence Within Each Finger
DRSHLTR

SEQ ID NO: 33 - protein, 7 aa, synthetic construct
  REGION 1..7: note = DNA-Recognition Helix Amino Acid Sequence Within Each Finger
DSSHRTR

SEQ ID NO: 34 - protein, 7 aa, synthetic construct
  REGION 1..7: note = DNA-Recognition Helix Amino Acid Sequence Within Each Finger
DRSNLTR

SEQ ID NO: 35 - protein, 7 aa, synthetic construct
  REGION 1..7: note = DNA-Recognition Helix Amino Acid Sequence Within Each Finger
DRSHLAR

SEQ ID NO: 36 - protein, 7 aa, synthetic construct
  REGION 1..7: note = DNA-Recognition Helix Amino Acid Sequence Within Each Finger
DRSYRNT

SEQ ID NO: 37 - protein, 7 aa, synthetic construct
  REGION 1..7: note = DNA-Recognition Helix Amino Acid Sequence Within Each Finger
RRSDLKR

SEQ ID NO: 38 - protein, 7 aa, synthetic construct
  REGION 1..7: note = DNA-Recognition Helix Amino Acid Sequence Within Each Finger
ERGTLAR

SEQ ID NO: 39 - protein, 7 aa, synthetic construct
  REGION 1..7: note = DNA-Recognition Helix Amino Acid Sequence Within Each Finger
DRSALAR

SEQ ID NO: 40 - protein, 7 aa, synthetic construct
  REGION 1..7: note = DNA-Recognition Helix Amino Acid Sequence Within Each Finger
DRSNLSR

SEQ ID NO: 41 - protein, 7 aa, synthetic construct
  REGION 1..7: note = DNA-Recognition Helix Amino Acid Sequence Within Each Finger
LKFALAN

SEQ ID NO: 42 - protein, 7 aa, synthetic construct
  REGION 1..7: note = DNA-Recognition Helix Amino Acid Sequence Within Each Finger
RSDNLST

SEQ ID NO: 43 - protein, 7 aa, synthetic construct
  REGION 1..7: note = DNA-Recognition Helix Amino Acid Sequence Within Each Finger
RSAALAR

SEQ ID NO: 44 - protein, 7 aa, synthetic construct
  REGION 1..7: note = DNA-Recognition Helix Amino Acid Sequence Within Each Finger
RSDHLST

SEQ ID NO: 45 - protein, 7 aa, synthetic construct
  REGION 1..7: note = DNA-Recognition Helix Amino Acid Sequence Within Each Finger
QSAHRIT

SEQ ID NO: 46 - protein, 7 aa, synthetic construct
  REGION 1..7: note = DNA-Recognition Helix Amino Acid Sequence Within Each Finger
RSDDLSK

SEQ ID NO: 47 - protein, 7 aa, synthetic construct
  REGION 1..7: note = DNA-Recognition Helix Amino Acid Sequence Within Each Finger
RSDHRTN

SEQ ID NO: 48 - protein, 7 aa, synthetic construct
  REGION 1..7: note = DNA-Recognition Helix Amino Acid Sequence Within Each Finger
TSANLSR

SEQ ID NO: 49 - protein, 7 aa, synthetic construct
  REGION 1..7: note = DNA-Recognition Helix Amino Acid Sequence Within Each Finger
RSDSLSR

SEQ ID NO: 50 - protein, 7 aa, synthetic construct
  REGION 1..7: note = DNA-Recognition Helix Amino Acid Sequence Within Each Finger
DRSVRTK

SEQ ID NO: 51 - protein, 7 aa, synthetic construct
  REGION 1..7: note = DNA-Recognition Helix Amino Acid Sequence Within Each Finger
YKHVLSD

SEQ ID NO: 52 - protein, 7 aa, synthetic construct
  REGION 1..7: note = DNA-Recognition Helix Amino Acid Sequence Within Each Finger
TSGSLTR

SEQ ID NO: 53 - protein, 7 aa, synthetic construct
  REGION 1..7: note = DNA-Recognition Helix Amino Acid Sequence Within Each Finger
RSDSLLR

SEQ ID NO: 54 - protein, 7 aa, synthetic construct
  REGION 1..7: note = DNA-Recognition Helix Amino Acid Sequence Within Each Finger
NYASRTW

SEQ ID NO: 55 - protein, 7 aa, synthetic construct
  REGION 1..7: note = DNA-Recognition Helix Amino Acid Sequence Within Each Finger
RSDSLSQ

SEQ ID NO: 56 - protein, 7 aa, synthetic construct
  REGION 1..7: note = DNA-Recognition Helix Amino Acid Sequence Within Each Finger
RKADRTR

SEQ ID NO: 57 - protein, 7 aa, synthetic construct
  REGION 1..7: note = DNA-Recognition Helix Amino Acid Sequence Within Each Finger
LKDTLRR

SEQ ID NO: 58 - protein, 7 aa, synthetic construct
  REGION 1..7: note = DNA-Recognition Helix Amino Acid Sequence Within Each Finger
RSANLAR

SEQ ID NO: 59 - protein, 7 aa, synthetic construct
  REGION 1..7: note = DNA-Recognition Helix Amino Acid Sequence Within Each Finger
QSSDLRR

SEQ ID NO: 60 - protein, 7 aa, synthetic construct
  REGION 1..7: note = DNA-Recognition Helix Amino Acid Sequence Within Each Finger
HKTSLKD

SEQ ID NO: 61 - protein, 7 aa, synthetic construct
  REGION 1..7: note = DNA-Recognition Helix Amino Acid Sequence Within Each Finger
QSNHLTE

SEQ ID NO: 62 - protein, 7 aa, synthetic construct
  REGION 1..7: note = DNA-Recognition Helix Amino Acid Sequence Within Each Finger
QNATRTK

SEQ ID NO: 63 - protein, 7 aa, synthetic construct
  REGION 1..7: note = DNA-Recognition Helix Amino Acid Sequence Within Each Finger
DRSALSR

SEQ ID NO: 64 - protein, 7 aa, synthetic construct
  REGION 1..7: note = DNA-Recognition Helix Amino Acid Sequence Within Each Finger
RSDHLSR

SEQ ID NO: 65 - protein, 7 aa, synthetic construct
  REGION 1..7: note = DNA-Recognition Helix Amino Acid Sequence Within Each Finger
LRHHLTR

SEQ ID NO: 66 - protein, 7 aa, synthetic construct
  REGION 1..7: note = DNA-Recognition Helix Amino Acid Sequence Within Each Finger
LRHNLRA

SEQ ID NO: 67 - protein, 7 aa, synthetic construct
  REGION 1..7: note = DNA-Recognition Helix Amino Acid Sequence Within Each Finger
ASKTRTN

SEQ ID NO: 68 - protein, 7 aa, synthetic construct
  REGION 1..7: note = DNA-Recognition Helix Amino Acid Sequence Within Each Finger
QSGDLTR

SEQ ID NO: 69 - protein, 7 aa, synthetic construct
  REGION 1..7: note = DNA-Recognition Helix Amino Acid Sequence Within Each Finger
DRSDLSR

SEQ ID NO: 70 - protein, 7 aa, synthetic construct
  REGION 1..7: note = DNA-Recognition Helix Amino Acid Sequence Within Each Finger
DRSYRNT

SEQ ID NO: 71 - protein, 7 aa, synthetic construct
  REGION 1..7: note = DNA-Recognition Helix Amino Acid Sequence Within Each Finger
ERGTLAR

SEQ ID NO: 72 - protein, 294 aa, synthetic construct
  REGION 1..294: note = polypeptide sequences
MAPKKKRKVGVPAAMAERPFQCRICMRNFSDRSDLSRHIRTHTGEKPFACDICGRKFAQSGSLTRHTKIHTGSQKPFQCRICMRNFSQSGDLTRHIRTHTGEKPFACDICGRKFADSSNRAKHTKIHTGSQKPFQCRICMRNFSDRSHLSRHIRTHTGEKPFACDICGRKFATSGHLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 73 - protein, 294 aa, synthetic construct
  REGION 1..294: note = polypeptide sequences
MAPKKKRKVGVPAAMAERPFQCRICMRNFSASKTRTNHIRTHTGEKPFACDICGRKFAQSGSLTRHTKIHTGSQKPFQCRICMRNFSQSGDLTRHIRTHTGEKPFACDICGRKFADSSNRAKHTKIHTGSQKPFQCRICMRNFSDRSHLTRHIRTHTGEKPFACDICGRKFATSGHLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 74 - protein, 265 aa, synthetic construct
  REGION 1..265: note = polypeptide sequences
MAPKKKRKVGVPAAMAERPFQCRICMRNFSASKTRTNHIRTHTGEKPFACDICGRKFAQSGSLTRHTKIHTGSQKPFQCRICMRNFSQSGDLTRHIRTHTGEKPFACDICGRKFADSSNRAKHTKIHTGEKPFQCRICMRKFADSSHRTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 75 - skipped

SEQ ID NO: 76 - protein, 265 aa, synthetic construct
  REGION 1..265: note = polypeptide sequences
MAPKKKRKVGVPAAMAERPFQCRICMRNFSASKTRTNHIRTHTGEKPFACDICGRKFAQSGSLTRHTKIHTGSQKPFQCRICMRNFSQSGDLTRHIRTHTGEKPFACDICGRKFADRSNLTRHTKIHTGEKPFQCRICMRKFADRSHLARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 77 - protein, 294 aa, synthetic construct
  REGION 1..294: note = polypeptide sequences
MAPKKKRKVGVPAAMAERPFQCRICMRNFSASKTRTNHIRTHTGEKPFACDICGRKFAQSGSLTRHTKIHTGSQKPFQCRICMRNFSQSGDLTRHIRTHTGEKPFACDICGRKFADRSNLTRHTKIHTGSQKPFQCRICMRNFSDRSHLSRHIRTHTGEKPFACDICGRKFATSGHLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 78 - protein, 302 aa, synthetic construct
  REGION 1..302: note = polypeptide sequences
MAPKKKRKVGVPAAMAERPFQCRICMRNFSDRSYRNTHIRTHTGEKPFACDICGRKFARRSDLKRHTKIHTHPRAPIPKPFQCRICMRNFSERGTLARHIRTHTGEKPFACDICGRKFADRSALARHTKIHTHPRAPIPKPFQCRICMRNFSDRSHLTRHIRTHTGEKPFACDICGRKFAQSGDLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 79 - protein, 294 aa, synthetic construct
  REGION 1..294: note = polypeptide sequences
MAPKKKRKVGVPAAMAERPFQCRICMRNFSDRSNLSRHIRTHTGEKPFACDICGRKFALKFALANHTKIHTGSQKPFQCRICMRNFSRSDNLSTHIRTHTGEKPFACDICGRKFARSAALARHTKIHTGSQKPFQCRICMRNFSRSDHLSTHIRTHTGEKPFACDICGRKFAQSAHRITHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 80 - protein, 294 aa, synthetic construct
  REGION 1..294: note = polypeptide sequences
MAPKKKRKVGVPAAMAERPFQCRICMRNFSRSDDLSKHIRTHTGEKPFACDICGRKFARSDHRTNHTKIHTGSQKPFQCRICMRNFSDRSHLTRHIRTHTGEKPFACDICGRKFATSANLSRHTKIHTGSQKPFQCRICMRNFSRSDSLSRHIRTHTGEKPFACDICGRKFADRSVRTKHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 81 - protein, 298 aa, synthetic construct
  REGION 1..298: note = polypeptide sequences
MAPKKKRKVGVPAAMAERPFQCRICMRNFSYKHVLSDHIRTHTGEKPFACDICGRKFATSGSLTRHTKIHTHPRAPIPKPFQCRICMRNFSRSDSLLRHIRTHTGEKPFACDICGRKFANYASRTWHTKIHTGSQKPFQCRICMRNFSRSDSLLRHIRTHTGEKPFACDICGRKFANYASRTWHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 82 - protein, 294 aa, synthetic construct
  REGION 1..294: note = polypeptide sequences
MAPKKKRKVGVPAAMAERPFQCRICMRNFSRSDSLSQHIRTHTGEKPFACDICGRKFARKADRTRHTKIHTGSQKPFQCRICMRNFSQSGDLTRHIRTHTGEKPFACDICGRKFALKDTLRRHTKIHTGSQKPFQCRICMRNFSRSANLARHIRTHTGEKPFACDICGRKFAQSSDLRRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 83 - protein, 265 aa, synthetic construct
  REGION 1..265: note = polypeptide sequences
MAPKKKRKVGVPAAMAERPFQCRICMRKFAHKTSLKDHTKIHTGEKPFQCRICMRNFSQSNHLTEHIRTHTGEKPFACDICGRKFAQNATRTKHTKIHTGSQKPFQCRICMRNFSDRSALSRHIRTHTGEKPFACDICGRKFARSDHLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 84 - protein, 294 aa, synthetic construct
  REGION 1..294: note = polypeptide sequences
MAPKKKRKVGVPAAMAERPFQCRICMRNFSQSGDLTRHIRTHTGEKPFACDICGRKFALKDTLRRHTKIHTGSQKPFQCRICMRNFSRSANLARHIRTHTGEKPFACDICGRKFAQSSDLRRHTKIHTGSQKPFQCRICMRNFSLRHHLTRHIRTHTGEKPFACDICGRKFALRHNLRAHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 85 - protein, 265 aa, synthetic construct
  REGION 1..265: note = full protein sequences
MAPKKKRKVGVPAAMAERPFQCRICMQNFSASKTRTNHIRTHTGEKPFACDICGRKFAQSGSLTRHTKIHTGSQKPFQCRICMQNFSQSGDLTRHIRTHTGEKPFACDICGRKFADRSNLTRHTKIHTGEKPFQCRICMRKFADRSHLARHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 86 - protein, 294 aa, synthetic construct
  REGION 1..294: note = full protein sequences
MAPKKKRKVGVPAAMAERPFQCRICMQNFSDRSDLSRHIRTHTGEKPFACDICGRKFAQSGSLTRHTKIHTGSQKPFQCRICMRNFSQSGDLTRHIRTHTGEKPFACDICGRKFADSSNRAKHTKIHTGSQKPFQCRICMRNFSDRSHLSRHIRTHTGEKPFACDICGRKFATSGHLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 87 - protein, 302 aa, synthetic construct
  REGION 1..302: note = full protein sequences
MAPKKKRKVGVPAAMAERPFQCRICMQNFSDRSYRNTHIRTHTGEKPFACDICGRKFARRSDLKRHTKIHTHPRAPIPKPFQCRICMQNFSERGTLARHIRTHTGEKPFACDICGRKFADRSALARHTKIHTHPRAPIPKPFQCRICMRNFSDRSHLTRHIRTHTGEKPFACDICGRKFAQSGDLTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 88 - protein, 265 aa, synthetic construct
  REGION 1..265: note = full protein sequences
MAPKKKRKVGVPAAMAERPFQCRICMQNFSASKTRTNHIRTHTGEKPFACDICGRKFAQSGSLTRHTKIHTGSQKPFQCRICMRNFSQSGDLTRHIRTHTGEKPFACDICGRKFADSSNRAKHTKIHTGEKPFQCRICMRKFADSSHRTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 89 - protein, 265 aa, synthetic construct
  REGION 1..265: note = full protein sequences
MAPKKKRKVGVPAAMAERPFQCRICMRNFSASKTRTNHIRTHTGEKPFACDICGRKFAQSGSLTRHTKIHTGSQKPFQCRICMQNFSQSGDLTRHIRTHTGEKPFACDICGRKFADSSNRAKHTKIHTGEKPFQCRICMRKFADSSHRTRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 90 - protein, 294 aa, synthetic construct
  REGION 1..294: note = full protein sequences
MAPKKKRKVGVPAAMAERPFQCRICMQNFSASKTRTNHIRTHTGEKPFACDICGRKFAQSGSLTRHTKIHTGSQKPFQCRICMQNFSQSGDLTRHIRTHTGEKPFACDICGRKFADRSNLTRHTKIHTGSQKPFQCRICMRNFSDRSHLSRHIRTHTGEKPFACDICGRKFATSGHLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 91 - protein, 294 aa, synthetic construct
  REGION 1..294: note = full protein sequences
MAPKKKRKVGVPAAMAERPFQCRICMQNFSASKTRTNHIRTHTGEKPFACDICGRKFAQSGSLTRHTKIHTGSQKPFQCRICMQNFSQSGDLTRHIRTHTGEKPFACDICGRKFADSSNRAKHTKIHTGSQKPFQCRICMRNFSDRSHLTRHIRTHTGEKPFACDICGRKFATSGHLSRHTKIHLRQKDAARGSGGDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVRS

SEQ ID NO: 92 - skipped

SEQ ID NO: 93 - protein, 7 aa, synthetic construct
  REGION 1..7: note = linker
TGEKPFQ

SEQ ID NO: 94 - protein, 8 aa, synthetic construct
  REGION 1..8: note = linker
TGGQRPFQ

SEQ ID NO: 95 - protein, 8 aa, synthetic construct
  REGION 1..8: note = linker
TGSQKPFQ

SEQ ID NO: 96 - protein, 8 aa, synthetic construct
  REGION 1..8: note = linker
TGSQRPFQ

SEQ ID NO: 97 - protein, 7 aa, synthetic construct
  REGION 1..7: note = linker
TGEKPFA

SEQ ID NO: 98 - protein, 11 aa, synthetic construct
  REGION 1..11: note = linker
TGGGGSQRPFQ

SEQ ID NO: 99 - protein, 11 aa, synthetic construct
  REGION 1..11: note = linker
TGGGGSQKPFQ

SEQ ID NO: 100 - protein, 12 aa, synthetic construct
  REGION 1..12: note = linker
THPRAPIPKPFQ

SEQ ID NO: 101 - protein, 12 aa, synthetic construct
  REGION 1..12: note = linker
TPNRRPAPKPFQ

SEQ ID NO: 102 - protein, 12 aa, synthetic construct
  REGION 1..12: note = linker
TVPRPTPPKPFQ

SEQ ID NO: 103 - protein, 12 aa, synthetic construct
  REGION 1..12: note = linker
TYPRPIAAKPFQ

SEQ ID NO: 104 - protein, 14 aa, synthetic construct
  REGION 1..14: note = linker
TGGGGSGGSQRPFQ

SEQ ID NO: 105 - protein, 14 aa, synthetic construct
  REGION 1..14: note = linker
TGGGGSGGSQKPFQ

SEQ ID NO: 106 - protein, 14 aa, synthetic construct
  REGION 1..14: note = linker
TLAPRPYRPPKPFQ

SEQ ID NO: 107 - protein, 16 aa, synthetic construct
  REGION 1..16: note = linker
TPGGKSSRTDRNKPFQ

SEQ ID NO: 108 - protein, 16 aa, synthetic construct
  REGION 1..16: note = linker
TPNPHRRTDPSHKPFQ

SEQ ID NO: 109 - protein, 6 aa, synthetic construct
  REGION 1..6: note = linker
LRGSGG

SEQ ID NO: 110 - protein, 21 aa, synthetic construct
  REGION 1..21: note = linker
GSGEGRGSLLTCGDVEENPGP

(19) United States
(12) Patent Application Publication
(10) Pub. No.: US 2026/0285923 A1
(43) Pub. Date: Sep. 24, 2026

(54) ZINC FINGER PROTEIN TRANSCRIPTION FACTORS FOR REPRESSING NAV1.7 EXPRESSION

(71) Applicant: Sangamo Therapeutics, Inc., Richmond, CA (US)

(72) Inventors: Mohammad Samie, Pleasant Hill, CA (US); Jason Eshleman, Berkeley, CA (US)

(73) Assignee: Sangamo Therapeutics, Inc., Richmond, CA (US)

(21) Appl. No.: 19/475,581

(22) PCT Filed: Apr. 17, 2024
(86) PCT No.: PCT/US2024/025043

§ 371 Date: Oct. 15, 2025

Related U.S. Application Data
(60) Provisional application No. 63/496,621, filed on Apr. 17, 2023.

(51) Int. Cl. C07K 14/47; A61K 38/00; A61K 48/00; A61P 25/02; C12N 15/86

(52) U.S. Cl. CPC C07K 14/47; A61K 48/0033; A61P 25/02; C12N 15/86; A61K 38/00; C07K 2319/81; C12N 2750/14143

(57) ABSTRACT

The present disclosure provides zinc finger fusion proteins that inhibit expression of Nav1.7 in the nervous system, and methods of using the proteins to treat pain disorders.

### CROSS REFERENCE TO RELATED APPLICATION

This application claims priority to, and the benefit of, U.S. Provisional Patent Application Ser. No. 63/496,621 filed on Apr. 17, 2023, entitled “Zinc Finger Protein Transcription Factors For Repressing NAV1.7 Expression,” the contents of which are incorporated by reference in their entirety.

### FIELD

This disclosure generally relates to zinc finger fusion proteins capable of repressing NAV1.7 expression.

### BACKGROUND OF THE INVENTION

Peripheral neuropathies are caused by severe damage to peripheral sensory or motor neurons. Damage to neurons is permanent, thus peripheral neuropathies are often chronic and painful. In particular, neuropathic pain is defined as pain that is caused by damage or alterations to peripheral sensory neurons. There is a broad array of heterogeneous pathologies that can potentially affect the peripheral sensory neurons such as surgical trauma, spinal cord injury, complex regional pain syndrome, nerve compression, vascular disease (e.g., stroke), neurological diseases (e.g., multiple sclerosis, syringomyelia), infectious diseases (e.g., HIV, leprosy, shingles), metabolic syndromes (e.g., diabetic mellitus, sarcoidosis, alcoholism), drugs (e.g., chemotherapeutics) and hereditary syndromes (e.g., Fabry's disease, erythromelalgia, channelopathies). Neuropathic pain is widely recognized as one of the most difficult pain syndromes to manage, and outcomes are often unsatisfactory. Given the high global unmet need and the lack of effective treatments for painful chronic neuropathies, there is an urgent need to develop therapeutics for the treatment of chronic neuropathic pain.

Voltage-gated sodium (Nav) channels are major molecular regulators of neuronal excitability in the central and peripheral nervous systems. To date, nine Nav channel isoforms (Nav1.1-Nav1.9) have been identified with various roles across the body. Of the nine known Nav channels in humans, Nav1.7 (SCN9A gene), Nav1.8 (SCN10A gene), and Nav1.9 (SCN11A gene) are implicated in pain transmission and enhanced hyperexcitability in nociceptive neurons (small fiber neurons that are involved with the transmission of pain signals to the brain) (Nau and Leipold, Neuroforum (2017) 23(3):A123-A130).

Among these three Nav channels, Nav1.7 has gained much attention in recent years due to its role in a spectrum of inherited human pain disorders. Loss-of-function mutations in the SCN9A gene are associated with a clinical condition called congenital insensitivity to pain (CIP), in which individuals exhibit complete loss of pain sensation (Cox et al., Nature (2006) 444:894-8). Alternately, gain-of-function mutations in the SCN9A gene have been linked to excessive pain in inherited erythromelalgia (IEM) and paroxysmal extreme pain disorder (PEPD), where individuals display recurrent, severe burning pain, redness, warmth, and often swelling of the distal extremities (Yong et al., J Med Genet. (2004) 41:171-4; Fertleman et al., Neuron (2006) 52:767-74). All these confirm the central role of Nav1.7 in the transduction and transmission of pain signals.

Nav1.7 regulates the transmission of pain signals to the brain following noxious stimuli by setting the thresholds for action potentials and amplifying small depolarizing inputs in the nociceptors in the dorsal root ganglion (DRG), acting as a molecular switch that regulates pain signals (Drenth and Waxman, J Clin Invest. (2007) 117:3603-9). Due to its role in pain sensation, Nav1.7 has emerged as a promising target for analgesic drug development. However, development of selective Nav1.7 small or large molecule inhibitors has been challenging, in part due to high amino acid and structural similarities among the Nav channels.

Given the significant role of Nav1.7 in manifestation of pain and the high unmet need for effective treatment of painful neuropathies, there is an urgent need for developing therapeutics that target this protein for the treatment of chronic neuropathic pain.

### SUMMARY OF THE INVENTION

The present disclosure provides a fusion protein comprising a zinc finger protein (ZFP) domain and a transcription repressor domain, wherein the ZFP domain binds to a target sequence of a human SCN9A gene, wherein the target sequence comprises any one of SEQ ID NOs: 16-24. In some embodiments, the target sequence is within 1.5 kb of a transcription start site (TSS) in the SCN9A gene, for example, within 1000 bps upstream of the TSS, and/or within 500 bps downstream of the TSS of the SCN9A gene.

In some embodiments, the ZFP domain comprises one or more (e.g., two, three, four, five, six, or more) zinc fingers, and/or the fusion protein represses expression of the SCN9A gene by at least about 40, 75, 90, 9%, or 99%, optionally with no or minimal detectable off-target binding or activity (e.g., binding to a gene that is not the SCN9A gene) detectable by a well-known method. Nonlimiting examples of DNA-binding recognition helix amino acid sequences of the present ZFPs are shown in Table 1 or Table 3. In some embodiments, the ZFP domain of the fusion protein comprises some or all of the DNA-binding recognition helix sequences as shown in a single row of Table 1 or Table 3.

In some embodiments, the transcription repressor domain of the present fusion proteins comprises a KRAB domain, wherein the KRAB domain optionally is from a human KOX1 protein. In some embodiments, the ZFP domain is linked to the transcription repressor domain through a peptide linker. In particular embodiments, the fusion protein comprises an amino acid sequence shown in Table 2 or Table 4.

In some embodiments, the ZFP domain of the fusion protein comprises five or six DNA-binding recognition helix sequences as shown in a single row of Table 1 or Table 3; binds to a target sequence shown in Table 1 or Table 3; comprises the DNA-binding recognition helix sequences of a ZFP transcription factor shown in Table 1 or Table 3; comprises the DNA-binding recognition helix sequences linked as shown in Table 1, Table 2, Table 3, or Table 4; and/or comprises an amino acid sequence selected from SEQ ID NOs: 25-92.

In some embodiments, the ZFP domain of the fusion protein comprises the recognition helix sequences and/or the amino acid sequence shown in a single row in Tables 1-4 for each of ZFP ID's 97028, 96986, or 96980.

In another aspect, the present disclosure provides a nucleic acid construct comprising a coding sequence for the present fusion protein, wherein the coding sequence is linked operably to a transcription regulatory element. In some embodiments, the transcription regulatory element is a mammalian promoter that is constitutively active or inducible in neurons. In some embodiments, the construct is a recombinant viral construct such as an adeno-associated viral (AAV) construct, an adenoviral construct, or a lentiviral construct.

In another aspect, the present disclosure provides a pharmaceutical composition comprising the present nucleic acid construct or the present recombinant viral construct, and a pharmaceutically acceptable carrier.

In another aspect, the present disclosure provides a host cell comprising the present nucleic acid construct or the present recombinant viral construct. The host cell may be a human cell, such as a neuronal cell or a pluripotent stem cell (e.g., an embryonic stem cell or an inducible pluripotent stem cell (iPSC)).

In another aspect, the present disclosure provides a method of inhibiting, deceasing, or repressing expression of Nav1.7 in a cell, preferably a human cell, by targeting a target site/sequence within or near a SCN9A gene with a zinc finger, TALE, or CRISPR repressor, thereby inhibiting, deceasing, or repressing expression of the gene. In some embodiments, the present disclosure provides a method of inhibiting, deceasing, or repressing expression of Nav1.7 in a cell, preferably a human cell, comprising expressing a zinc finger, TALE, or CRISPR repressor in the cell, wherein the zinc finger, TALE, or CRISPR repressor targets a target site/sequence within or near a SCN9A gene of the cell, thereby inhibiting, deceasing, or repressing expression of the SCN9A gene. In some embodiments, the target sequence is within 1.5 kb of a transcription start site (TSS) in the SCN9A gene. In some embodiments, the target sequence is within 1000 bps upstream of the TSS, and/or within 500 bps downstream of the TSS of the SCN9A gene. In some embodiments, the target sequence comprises at least 8 contiguous bps of any of SEQ ID NOs: 16-24. In some embodiments, the target sequence comprises at least 8 non-contiguous bps of any of SEQ ID NOs: 16-24. In some embodiments, the target sequence comprises any one of SEQ ID NOs: 16-24. In some embodiments, the target sequence is targeted by a zinc finger-repressor (ZFR) as described herein (e.g., Tables 1-4).

In another aspect, the present disclosure provides a method of inhibiting expression of Nav1.7 in a human neuron, comprising introducing into the neuron (e.g., a nociceptive neuron in the dorsal root ganglia (DRG)) the present fusion protein, optionally through introduction of a nucleic acid construct or a recombinant virus (e.g., AAV) encoding the fusion protein, thereby inhibiting the expression of Nav1.7 in the neuron. In some embodiments, the human neuron is in the body of a human patient.

In one aspect, the present disclosure provides a method of treating a pain disorder in a patient in need thereof, comprising administering to the patient the recombinant AAV or nucleic acid construct herein. In some embodiments, the recombinant AAV or nucleic acid construct is introduced to the patient via an intravenous, intrathecal, intracerebral, intracerebroventricular, intra-cisternal magna, epidural, oral, or intranasal route.

In the treatment methods herein, the patient is suffering from or at risk of developing, for example, a peripheral neuropathic pain disorder such as inherited erythromelalgia, paroxysmal extreme pain disorder, small fiber neuropathy (e.g., idiopathic small fiber neuropathy, small fiber neuropathy plus prediabetes, or diabetic small fiber neuropathy), large fiber neuropathy, trigeminal neuralgia, post-herpetic neuralgia, and painful diabetic neuropathy.

In the treatment methods herein, the recombinant AAV may be, for example, AAV9 or a pseudotype AAV derived from AAV9.

Also provided herein are fusion proteins, nucleic acid constructs, a recombinant virus, or a pharmaceutical composition, for use in a treatment method described herein, and the use of the fusion protein, nucleic acid construct, or recombinant virus for the manufacture of a medicament for use in the treatment method.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE DRAWINGS

FIG. 1 is a diagram illustrating specific targeting of the SCN9A gene by an engineered zinc finger protein-transcription factor (ZFP-TF), for example a zinc finger protein-repressor (ZF-R or ZFR). Binding of the ZFP-TF to the gene leads to reduced SCN9A transcription, which in turn leads to reduced SCN9A mRNA and Nav1.7 protein levels.

FIG. 2 is a panel of graphs showing the mouse Scn9a-repressing activity of 10 ZFP-TFs selected from a library of 288 ZFP-TFs designed to specifically target the mouse Scn9a gene. The y-axis in each graph is mouse Scn9a mRNA expression normalized to the geometric mean of two housekeeping genes (Atp5b and Eif4a2) and assessed 20 hours after transfection with mRNA coding for the different ZFP-TFs in Neuro 2A (N2A) mouse neuronal cells. The mRNA dose increases from left to right (30, 100, 300 ng). The bars represent the mean of four technical replicates and the error bars represent standard deviation. The numbers below the graphs are the internal reference numbers for the ZFP-TFs. An enlarged version of the titration scale is shown at the lower right of the figure. The blue bars represents the non-treated control in each panel.

FIG. 3 is a panel of graphs representing the top 9 ZFP-TFs selected for the target engagement study in animals after several rounds of optimization. For each ZFP-TF panel, on the left, volcano/scatter plots of Affymetrix/microarray data show changes in the transcriptomes of primary mouse cortical neurons 7 days post transduction with ZFP-TFs with AAV6 serotype. Cells were transduced at 3E3 MOI. Numbers shown in red and green indicate the counts of downregulated and upregulated off-target genes, respectively; red circles represent downregulated off-target genes and green circles represent upregulated off-target genes. Data were derived from at least two independent experiments and six biological replicates per experiment. There is no repression of the Scn9a gene (exhibited in the volcano plots) since primary mouse cortical neurons do not express the Nav1.7 protein. On the right, is a panel of graphs showing the mouse Scn9a-repressing activity of the ZFP-TFs in Neuro 2A (N2A) mouse neuronal cell lines. The dark blue bars in the bottom set of graphs represent untreated cells.

FIG. 4 is a panel of graphs showing ZFP-TF and Scn9a mRNA expression levels following intrathecal-lumbar (IT-L) delivery of AAV9 encoding the top 9 ZFP-TFs in lumbar, cervical, and thoracic DRGs of C57BL/6 mice. The y-axis in each graph is absolute or normalized mRNA expression levels of ZFP-TFs or mouse Scn9a, respectively. Data were collected from combining three pairs of DRGs from each level at four weeks following treatment with AAV9 (dose-2E11 vg/mouse). ZFP-TFs are encoded under the human synapsin 1 promoter. Vehicle treatment was used as negative control. The ZFP-TFs used are indicated on the x-axis. The bars represent the mean of values from eight mice and the error bars represent standard deviation.

FIG. 5 is a panel of graphs showing neuronal marker NeuN, and neuroinflammatory markers (Iba1 and Gfap) mRNA expression levels following intrathecal-lumbar (IT-L) delivery of AAV9 encoding the top 9 ZFP-TFs in lumbar DRGs of C57BL/6 mice. The y-axis in each graph is normalized mRNA expression levels of the markers. Data were collected from combining three pairs of DRGs from lumbar regions at four weeks following treatment with AAV9 (dose—2E11 vg/animal). ZFP-TFs are encoded under the human synapsin 1 promoter. Vehicle treatment was used as negative control. The ZFP-TFs used are indicated on the x-axis. The bars represent the mean of values from eight mice and the error bars represent standard deviation.

FIG. 6A Schematic diagram illustrating the spared nerve injury (SNI) model. SNI model is made surgically by damaging two of the three terminal branches of the sciatic nerve (tibial and common peroneal nerves) leaving the sural nerve intact. FIG. 6B Overview and timeline of the pain efficacy study using the SNI mouse model. Mechanical and cold allodynia were measured in WT mice before they go under SNI surgery. 7 days after the SNI surgery (days 0), mechanical and cold allodynia were measured again, and animals were injected with AAV9-hSyn1-ZFP-TF at 8E11 vg/animal. After four weeks, pain responses were measured again, and animals were sacrificed and DRGs were collected for gene expression and pathological analysis.

FIGS. 7A-7B are panels of graphs showing (FIG. 7A) Scn9a and (FIG. 7B) ZFP-TF mRNA expression levels following intrathecal-lumbar (IT-L) delivery of AAV9 encoding four representative ZFP-TFs used in the neuropathic assessment pain study. ZFP-TF and Scn9a mRNA expression levels were assessed in DRGs collected from lumbar, cervical, and thoracic levels on day 29 of the experiment from both male and female mice. The y-axis in each graph is absolute or normalized mRNA expression levels of ZFP-TFs or mouse Scn9a, respectively. Data are collected from combining three pairs of DRGs from each level following treatment with AAV9-ZFP-TF. ZFP-TFs are encoded under the human synapsin 1 promoter. Vehicle treatment was used as negative control. The ZFP-TFs used are indicated on the x-axis. The bars represent the mean of values from six mice and the error bars represent standard deviation.

FIG. 8 Microscopy images of whole lumbar DRG are presented following RNAscope in situ hybridization in combination with immunohistochemistry to assess the Scn9a mRNA levels within nociceptors following AAV9-ZFP-TF (95676) treatment on a single cell level 4 weeks post injection. Peripherin specific antibody was used to identify nociceptors (green) in combination with RNA probes against Scn9a (white) and ZFP-TF (red).

FIG. 9 is a panel of graphs showing female and male mouse response to mechanical allodynia at different timepoints following different treatments in SNI pain model. Mechanical induced pain responses were measured at day −7 (healthy mice) and then at day 0 (7 days post SNI surgery). 28 days after IT-L injection of AAV9-ZFP-TFs (94091, 95664, 95676, and 95807), mechanical allodynia was measured again to evaluate efficacy. Gabapentin (GBP) was injected on day 28 one hour before pain measurements. Dots represent individual animal; n=7-8; error bars are SEM

FIG. 10 is a panel of graphs showing female and male mouse response to cold allodynia at different timepoints following different treatments in SNI pain model. Cold induced pain responses were measured at day −7 (healthy mice) and then at day 0 (7 days post SNI surgery). 28 days after IT-L injection of AAV9-ZFP-TFs (94091, 95664, 95676, and 95807), cold allodynia was measured again to evaluate efficacy. Gabapentin (GBP) was injected on day 28 one hour before pain measurements. Dots represent individual animal; n=7-8; error bars are SEM

FIG. 11 is a panel of graphs showing the human SCN9A-repressing activity of 12 ZFP-TFs selected from a library of 384 ZFP-TFs designed to specifically target human/NHP SCN9A gene in neuronal cell lines. The y-axis in each graph is human SCN9A mRNA expression normalized to the geometric mean of two housekeeping genes (Atp5b and Eif4a2) and assessed 20 hours after transfection with mRNA coding for the different ZFP-TFs in SK-N-MC human neuronal cells. The mRNA dose increases from left to right (3, 10, 30, 100, 300 ng). The bars represent the mean of four technical replicates and the error bars represent standard deviation. The numbers below the graphs are the internal reference numbers for the ZFP-TFs. An enlarged version of the titration scale is shown at the lower right of the figure. The orange graph represents the non-treated control in each panel.

FIG. 12 is a panel of graphs showing the human SCN9A-repressing activity of representative ZFP-TFs in human iPSC-derived GABAergic neurons For each panel, on the right, the graph represents RT-qPCR analysis of SCN9A expression following ZFP-TF treatment. The y-axis is SCN9A mRNA expression normalized to the geometric mean of two housekeeping genes (Atp5b and Eif4A2) and assessed 32 days after transduction with AAV6 for the different ZFP-TFs in human iPSC-derived GABAergic neurons and. The amount of AAV6 used is indicated in the legend on the lower right, with the AAV6 dose increasing from left to right (MOI of 3E3, 1E4, 3E4, 1E5, and 3E5). The bars represent the mean of four technical replicates and the error bars represent standard deviation. The blue represents non-treated control. On the left, volcano/scatter plots of Affymetrix/microarray data show changes in the transcriptomes of human iPSC-derived GABAergic neurons 32 days post transduction with ZFP-TFs with AAV6 serotype. Cells were transduced at 1E5 MOI. Numbers shown in red and green indicate the counts of downregulated and upregulated off-target genes, respectively; red circles represent downregulated off-target genes and green circles represent upregulated off-target genes. Data were derived from at least two independent experiments and six biological replicates per experiment.

FIG. 13 is a panel of graphs showing the repressive activity of representative ZFP-TFs in human progenitor sensory neurons. The expression levels of different SCN genes were measured (SCN1A (Nav1.1), SCN2A (Nav1.2), SCN3A (Nav1.3), SCN8A (Nav1.6), SCN9A (Nav1.7), SCN10a (Nav1.8), and SCN11a (Nav1.9)). The y-axis is SCNx mRNA expression normalized to the geometric mean of two housekeeping genes (Atp5b and Eif4a2) and assessed 7 days after transduction with AAV6 for the different ZFP-TFs in human progenitor sensory neurons. The amount of AAV6 used is indicated in the legend on the lower right, with the AAV6 dose increasing from left to right (MOI of 3E4, 1E5, and 3E5). The bars represent the mean of four technical replicates and the error bars represent standard deviation.

FIG. 14 is a panel of graphs showing ZFP-TF mRNA expression levels following intrathecal-lumbar (IT-L) delivery of AAV9 encoding the top 10 ZFP-TFs targeting human SCN9A gene in lumbar, cervical, and thoracic DRGs of C57BL/6 mice. The y-axis in each graph represents mRNA expression levels of the respective ZFP-TF. Data were collected from combining three pairs of DRGs from each level at four weeks following treatment with AAV9 (dose—2E11 vg/mouse). The ZFP-TFs are encoded under the human synapsin 1 promoter. Vehicle treatments were used as negative controls. The ZFP-TFs used are indicated on the x-axis. The bars represent the mean of values from five mice and the error bars represent standard deviation.

FIG. 15 is a panel of graphs showing the neuronal marker NeuN, and neuroinflammatory markers (Iba1 and Gfap) mRNA expression levels following intrathecal-lumbar (IT-L) delivery of AAV9 encoding the top 10 ZFP-TFs in lumbar, cervical, and thoracic DRGs of C57BL/6 mice. The y-axis in each graph is normalized mRNA expression levels of the markers. Data were collected from combining three pairs of DRGs from each level at four weeks following treatment with AAV9 (dose—2E11 VG per animal). The ZFP-TFs are encoded under the human synapsin 1 promoter. Vehicle treatments were used as negative controls. The ZFP-TFs used are indicated on the x-axis. The bars represent the mean of values from five mice and the error bars represent standard deviation.

FIG. 16 Overview and timeline of the nonhuman primate (NHP) study evaluating the potency and safety of the selected ZFP-TFs (97028, 96986, and 96980) targeting human/NHP SCN9A gene in Cynomolgus monkeys.

FIG. 17 is a panel of graphs showing ZFP-TF mRNA expression levels following intrathecal-lumbar (IT-L) delivery of AAV9 constructs encoding ZFP-TFs (97028, 96986, and 96980) targeting human/NHP SCN9A gene. ZFP-TF mRNA expression levels were assessed in NHP DRGs collected from lumbar, cervical, and thoracic levels on day 29 of the experiment. ZFP-TFs are encoded under the human synapsin 1 promoter. Vehicle treatment group was used as negative control. The ZFP-TFs used are indicated on the x-axis. DRGs collected from different levels for each region were combined and the average copy/ng RNA is presented. mean±s.e.m

FIG. 18 is a panel of graphs showing SCN9A mRNA expression levels following intrathecal-lumbar (IT-L) delivery of AAV9 constructs encoding ZFP-TFs (97028, 96986, and 96980) targeting human/NHP SCN9A gene. SCN9A mRNA expression levels were assessed in NHP DRGs collected from lumbar, cervical, and thoracic levels on day 29 of the experiment. ZFP-TFs are encoded under the human synapsin 1 promoter. Vehicle treatment group was used as negative control. The ZFP-TFs used are indicated on the x-axis. DRGs collected from different levels for each region were combined and the average copy/ng RNA is presented. mean±s.e.m.

FIG. 19 panels represent the total number of histopathological findings in all DRG levels (lumbar, thoracic, and cervical) following intrathecal-lumbar (IT-L) delivery of AAV9 constructs encoding ZFP-TFs (97028, 96986, and 96980) targeting human/NHP SCN9A gene. Histopathological findings are categorized based severity of the incidents (minimal, mild, moderate, marked, and severe) and dose (Low Dose—1E11, Mid Dose—1E13, and High Dose—9E13) that they occurred.

FIG. 20 panels represent the incident of histopathological findings for each DRG level following intrathecal-lumbar (IT-L) delivery of AAV9 constructs encoding ZFP-TFs (97028, 96986, and 96980) targeting human/NHP SCN9A gene. Histopathological findings are categorized based severity of the incidents (minimal, mild, moderate, marked, and severe) and dose (Low Dose—1E11, Mid Dose—1E13, and High Dose—9E13) that they occurred. In addition, the data are represented based on the type of histopathological findings: mononuclear cell infiltration (MN) and neuronal degeneration.

### DETAILED DESCRIPTION OF THE INVENTION

The present disclosure provides ZFP domains that target sites (i.e., bind DNA sequences) in or near the human SCN9A gene. A ZFP domain as described herein may be attached or fused to another functional molecule or domain. For example, the ZFP domains of the present disclosure may be fused to a transcription factor to form a zinc finger protein-transcription factor (ZFP-TF). The transcription factor of such a ZFP-TF may specifically target the human SCN9A gene and repress its transcription into RNA. Reducing the level of Nav1.7 by introducing the ZFP-TFs into the DRG of a patient is expected to inhibit (e.g., reduce or stop) the transmission of pain signals. The present ZFP-TFs thus can be used for the prevention and/or treatment of pain disorders such as peripheral neuropathies.

The present ZFP-TF approach to Nav1.7 inhibition has several advantages over the current approaches being tested by others, which include administration of (i) antisense oligonucleotides (ASOs) that bind Nav1.7 mRNA and prevent its translation and (ii) immunotherapeutic anti-Nav1.7 antibodies. ZFP-TFs may need to be administered only once (by introducing to the patient a ZFP-TF expression construct), while ASOs require repeated dosing. In addition, the ZFP-TF approach only needs to engage the two alleles of the SCN9A gene in the genome of each cell. By contrast, ASOs need to engage numerous copies of the SCN9A mRNA in each cell. Additionally, the distribution and tropism of ASOs is fixed, whereas the ZFP-TF approach can be targeted to different cell types and nervous system regions by altering the promoter, serotype, and route of administration.

The present ZFP-TF approach is also advantageous over the antibody approach. Development of selective Nav1.7 antibody inhibitors has been challenging due to high amino acid and structural similarities among the Nav channels. To date, nine Nav channel isoforms (Nav1.1-Nav1.9) have been identified with various roles across the body, and they all share very close sequence homology at the protein level, making it difficult to design specific therapeutic antibodies against any one of them.

### I. Targets of the ZFP Domains

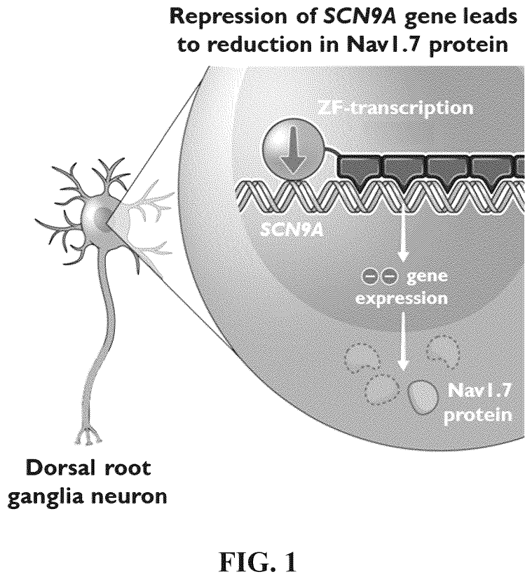

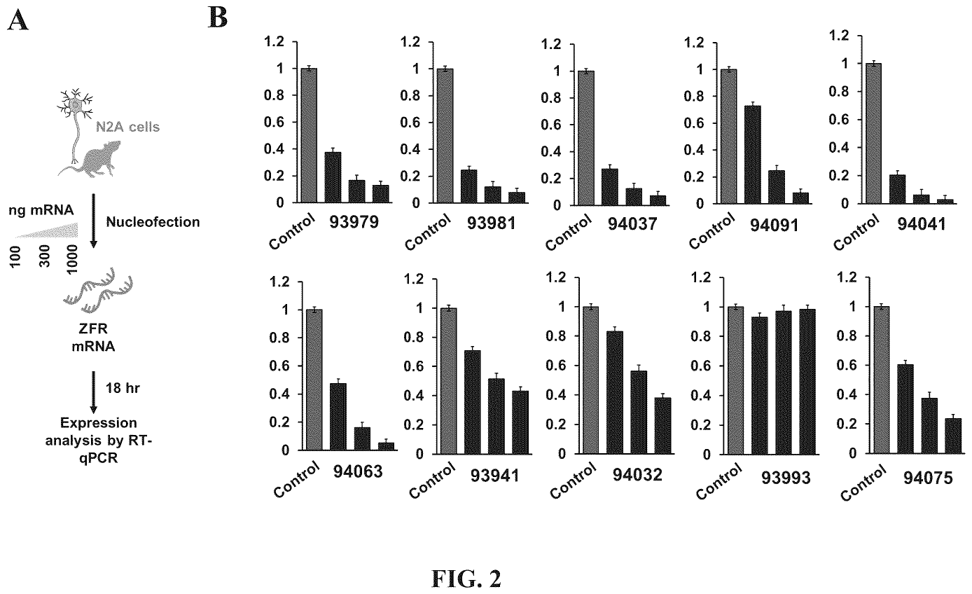

The ZFP domains of the present fusion proteins bind specifically to a target region in or near the human SCN9A gene. FIG. 1 illustrates the binding of a ZFP domain to a DNA sequence in the SCN9A gene.

The human SCN9A gene, also known as PN1, Nav1.7, Nav 1.7, sodium voltage-gated channel alpha subunit 9, and voltage-gated sodium channel subunit alpha NaV1.7, spans about 114 kb and has been mapped to chr2q24.3:166,375,987-166, 195,185 (GRCh38.p14). Its nucleotide sequence is available at GenBank accession number ENSG00000169432. The SCN9A gene comprises 27 exons. The full-length, canonical isoform 1 of human Nav1.7 protein has the following amino acid sequence (UniProt. No. Q15858-1):

(SEQ ID NO: 1)
MAMLPPPGPQSFVHFTKQSLALIEQRIAERKSKEPKEEKKDDDEEAPKP
SSDLEAGKQLPFIYGDIPPGMVSEPLEDLDPYYADKKTFIVLNKGKTIF
RFNATPALYMLSPFSPLRRISIKILVHSLFSMLIMCTILTNCIFMTMNN
PPDWTKNVEYTFTGIYTFESLVKILARGFCVGEFTFLRDPWNWLDFVVI
VFAYLTEFVNLGNVSALRTFRVLRALKTISVIPGLKTIVGALIQSVKKL
SDVMILTVFCLSVFALIGLQLFMGNLKHKCFRNSLENNETLESIMNTLE
SEEDFRKYFYYLEGSKDALLCGFSTDSGQCPEGYTCVKIGRNPDYGYTS
FDTFSWAFLALFRLMTQDYWENLYQQTLRAAGKTYMIFFVVVIFLGSFY
LINLILAVVAMAYEEQNQANIEEAKQKELEFQQMLDRLKKEQEEAEAIA
AAAAEYTSIRRSRIMGLSESSSETSKLSSKSAKERRNRRKKKNQKKLSS
GEEKGDAEKLSKSESEDSIRRKSFHLGVEGHRRAHEKRLSTPNQSPLSI
RGSLFSARRSSRTSLFSFKGRGRDIGSETEFADDEHSIFGDNESRRGSL
FVPHRPQERRSSNISQASRSPPMLPVNGKMHSAVDCNGVVSLVDGRSAL
MLPNGQLLPEVIIDKATSDDSGTTNQIHKKRRCSSYLLSEDMLNDPNLR
QRAMSRASILTNTVEELEESRQKCPPWWYRFAHKFLIWNCSPYWIKFKK
CIYFIVMDPFVDLAITICIVLNTLFMAMEHHPMTEEFKNVLAIGNLVFT
GIFAAEMVLKLIAMDPYEYFQVGWNIFDSLIVTLSLVELFLADVEGLSV
LRSFRLLRVFKLAKSWPTLNMLIKIIGNSVGALGNLTLVLAIIVFIFAV
VGMQLFGKSYKECVCKINDDCTLPRWHMNDFFHSFLIVFRVLCGEWIET
MWDCMEVAGQAMCLIVYMMVMVIGNLVVLNLFLALLLSSFSSDNLTAIE
EDPDANNLQIAVTRIKKGINYVKQTLREFILKAFSKKPKISREIRQAED
LNTKKENYISNHTLAEMSKGHNFLKEKDKISGFGSSVDKHLMEDSDGQS
FIHNPSLTVTVPIAPGESDLENMNAEELSSDSDSEYSKVRLNRSSSSEC
STVDNPLPGEGEEAEAEPMNSDEPEACFTDGCVWRFSCCQVNIESGKGK
IWWNIRKTCYKIVEHSWFESFIVLMILLSSGALAFEDIYIERKKTIKII
LEYADKIFTYIFILEMLLKWIAYGYKTYFTNAWCWLDFLIVDVSLVTLV
ANTLGYSDLGPIKSLRTLRALRPLRALSRFEGMRVVVNALIGAIPSIMN
VLLVCLIFWLIFSIMGVNLFAGKFYECINTTDGSRFPASQVPNRSECFA
LMNVSQNVRWKNLKVNFDNVGLGYLSLLQVATFKGWTIIMYAAVDSVNV
DKQPKYEYSLYMYIYFVVFIIFGSFFTLNLFIGVIIDNFNQQKKKLGGQ
DIFMTEEQKKYYNAMKKLGSKKPQKPIPRPGNKIQGCIFDLVTNQAFDI
SIMVLICLNMVTMMVEKEGQSQHMTEVLYWINVVFIILFTGECVLKLIS
LRHYYFTVGWNIFDFVVVIISIVGMFLADLIETYFVSPTLFRVIRLARI
GRILRLVKGAKGIRTLLFALMMSLPALFNIGLLLFLVMFIYAIFGMSNF
AYVKKEDGINDMFNFETFGNSMICLFQITTSAGWDGLLAPILNSKPPDC
DPKKVHPGSSVEGDCGNPSVGIFYFVSYIIISFLVVVNMYIAVILENFS
VATEESTEPLSEDDFEMFYEVWEKFDPDATQFIEFSKLSDFAAALDPPL
LIAKPNKVQLIAMDLPMVSGDRIHCLDILFAFTKRVLGESGEMDSLRSQ
MEERFMSANPSKVSYEPITTTLKRKQEDVSATVIQRAYRRYRLRQNVKN
ISSIYIKDGDRDDDLLNKKDMAFDNVNENSSPEKTDATSSTTSPPSYDS
VTKPDKEKYEQDRTEKEDKGKDSKESKK

The DNA-binding ZFP domains of the ZFP-TFs direct the fusion proteins to a target region of the SCN9A gene and bring the transcriptional repression domains of the fusion proteins to the target region. The repression domains recruit transcriptional co-repressor complexes to modify the chromatin into a non-permissive state for transcription by RNA Polymerase II. The target region for the ZFP-TFs can be any suitable site in or near the SCN9A gene that allows repression of gene expression. By way of example, the target region includes, or is adjacent to (either downstream or upstream of) a SCN9A transcription start site (TSS) or a SCN9A transcription regulatory element (e.g., promoter, enhancer, RNA polymerase pause site, and the like). In some embodiments, the transcription regulatory element comprises a mammalian promoter.

In some embodiments, the genomic target region is at least 8 bps in length. For example, the target region may be 8 bps to 40 bps in length, such as 12, 15, 16, 17, 18, 19, 20, 21, 24, 27, 30, 33, or 36 bps in length. The targeted sequence may be on the sense strand of the gene, or the antisense strand of the gene. To ensure targeting accuracy and to reduce off-target binding or activity by the ZFP-TFs, the sequence of the selected SCN9A target region preferably has less than 75% homology (e.g., less than 70%, less than 65%, less than 60%, or less than 50%) to sequences in other genes. In certain embodiments, the target region of the present ZFP-TFs is 12-20 (e.g., 12-18, 15-19, 15, 18, or 19) bps in length and resides within 1500 bps upstream to 1000 bps downstream (e.g.,-1000 bps to +1000 bps, +750, or +500 bps) of the TSS. In some embodiments, the target region is within 1000 bps upstream of the TSS. In some embodiments, the target region is within 500 bps downstream of the TSS.

In some embodiments, the present engineered ZFPs bind to a target site (i.e., Target Sequence) as shown in a single row of Table 1 or Table 3, including contiguous or non-contiguous sequences within these target sites, preferably with no or little detectable off-target binding or activity. In some embodiments, the Target Sequence comprises and/or is within any one of SEQ ID NOs: 16-24. In some embodiments, the Target Sequence comprises at least 8 contiguous bps of any of SEQ ID NOs: 16-24 (i.e., the Target Sequences shown in Table 1 or Table 3). In some embodiments, the Target Sequence comprises at least 8 non-contiguous bps of any of SEQ ID NOs: 16-24 (i.e., the Target Sequences shown in Table 1 or Table 3). In some embodiments, the Target Sequence comprises any one of SEQ ID NOs: 16-24 (i.e., the Target Sequences shown in Table 1 or Table 3).

In some embodiments, binding of the engineered ZFPs to any of the Target Sequences disclosed herein results in repression of expression of the SCN9A gene by at least about 40%, about 75%, about 90%, about 95%, or about 99%.

Other criteria for further evaluating target segments include the prior availability of ZFPs binding to such segments or related segments, ease of designing new ZFPs to bind a given target segment, and/or off-target binding risk.

### II. Zinc Finger Protein Domains

A “zinc finger protein” or “ZFP” refers to a protein having a DNA-binding domain that is stabilized by zinc. ZFPs bind to DNA in a sequence-specific manner. The individual DNA-binding unit of a ZFP is referred to as a “zinc finger.” Each finger contains a DNA-binding “recognition helix” that is typically comprised of seven amino acid residues and determines DNA binding specificity. A ZFP domain has at least one finger and each finger binds from two to four base pairs of nucleotides, typically three or four base pairs of DNA (contiguous or noncontiguous). Each zinc finger typically comprises approximately 28-30 amino acids and chelates zinc. An engineered ZFP can have a novel binding specificity, compared to a naturally occurring ZFP. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers that bind the particular triplet or quadruplet sequence. See, e.g., ZFP design methods described in detail in U.S. Pat. Nos. 5,789,538; 5,925,523; 6,007,988; 6,013,453; 6,140,081; 6,200,759; 6,453,242; 6,534,261; 6,979,539; and 8,586,526; and International Patent Publications WO 95/19431; WO 96/06166; WO 98/53057; WO 98/53058; WO 98/53059; WO 98/53060; WO 98/54311; WO 00/27878; WO 01/60970; WO 01/88197; WO 02/016536; WO 02/099084; and WO 03/016496. A ZFP domain as described herein may be attached or fused to another molecule (e.g., domain), for example, a protein. Such ZFP-fusions may comprise a domain that enables gene activation (e.g., activation domain), gene repression (e.g., repression domain), ligand binding (e.g., ligand-binding domain), high-throughput screening (e.g., ligand-binding domain), localized hypermutation (e.g., activation-induced cytidine deaminase domain), chromatin modification (e.g., histone deacetylase domain), recombination (e.g., recombinase domain), targeted integration (e.g., integrase domain), DNA modification (e.g., DNA methyl-transferase domain), base editing (e.g., base editor domain), or targeted DNA cleavage (e.g., nuclease domain). In particular embodiments, a ZFP-fusion as described herein comprises a transcription factor domain. Examples of engineered ZFP domains are shown in Tables 1 to 4 below.

Table 1 shows exemplary ZFPs of the present disclosure that target human SCN9A. Shown in capital letters are the genomic target sequences (i.e., bound sequences) of the DNA-binding recognition helix sequences that are shown in a single row for each five or six finger ZFP shown (i.e., F1-F5, F1-F6, or F1 and F3-F6). Illustrative peptide linker sequences as shown in Table 5 between zinc fingers and between the ZFP domain and the repressor domain for each ZFP are also shown (i.e., L1, L2, L3, L4, L5, or L6). Numbers in parentheses indicate SEQ ID NOs.

Table 2 shows illustrative polypeptide sequences for the ZFP-TFs shown in Table 1. DNA-binding recognition helix sequences are in boldface. Zinc finger linkers are underlined, whereas interdomain linkers are double underlined.

Table 3 shows exemplary R→Q (Arg→Gln) variants of representative ZFP-TFs targeting the human SCN9A gene. Shown in capital letters are the genomic target sequences (i.e., bound sequences) of the DNA-binding recognition helix sequences that are shown in a single row for each five or six finger ZFP shown (i.e., F1-F5 or F1-F6). Table 3 also indicates illustrative peptide linker sequences as shown in Table 5 between zinc fingers and between the ZFP domain and the repressor domain for each ZFP shown (i.e., L1, L2, L3, L4, L5, or L6). The symbol “\hat{ }” indicates that the arginine (R) residue at the 4th position upstream of the 1st amino acid in the indicated finger is changed to glutamine (Q). Numbers in parentheses indicate SEQ ID NOs.

Table 4 shows illustrative full protein sequences for R→Q (Arg->Gln) variants of the ZFP-TFs shown in Table 3. DNA-binding recognition helix sequences are in boldface. Zinc finger linkers are underlined, whereas interdomain linkers are double underlined.

TABLE 1

| ZFP | Target Sequence | DNA-Recognition Helix Amino Acid Sequence Within Each Finger | | | | | | | | | | | |
|---|---|---|---|---|---|---|---|---|---|---|---|---|---|
| ID | (capital letters) | F1 | L1 | F2 | L2 | F3 | L3 | F4 | L4 | F5 | L5 | F6 | L6 |
| 93598 | gtGGTGGCGACGCTGTA | DRSDLSR | 0 | QSGSLTR | 0c | QSGDLTR | 0 | DSSNRAK | 0c | DRSHLSR | 0 | TSGHLSR | C1 |
| | GCCtctgcacc | (25) | | (26) | | (27) | | (28) | | (29) | | (30) | |
| | (16) | | | | | | | | | | | | |
| 93600 | gtGGTGGCGACGCTGTA | ASKTRTN | 0 | QSGSLTR | 0c | QSGDLTR | 0 | DSSNRAK | 0c | DRSHLTR | 0 | TSGHLSR | C1 |
| | GCCtctgcacc | (31) | | (26) | | (27) | | (28) | | (32) | | (30) | |
| | (16) | | | | | | | | | | | | |
| 93591 | gtGGCGACGCTGTAGCC | ASKTRTN | 0 | QSGSLTR | 0c | QSGDLTR | 0 | DSSNRAK | 0a | DSSHRTR | | | C1 |
| | tctgcaccatg | (31) | | (26) | | (27) | | (28) | | (33) | | | |
| | (17) | | | | | | | | | | | | |
| 93590 | gtGGCGACGCTGTAGCC | ASKTRTN | 0 | QSGSLTR | 0c | QSGDLTR | 0 | DRSNLTR | 0a | DRSHLAR | | | C1 |
| | tctgcaccatg | (31) | | (26) | | (27) | | (34) | | (35) | | | |
| | (17) | | | | | | | | | | | | |
| 93599 | gtGGTGGCGACGCTGTA | ASKTRTN | 0 | QSGSLTR | 0c | QSGDLTR | 0 | DRSNLTR | 0c | DRSHLSR | 0 | TSGHLSR | C1 |
| | GCCtctgcacc | (31) | | (26) | | (27) | | (34) | | (29) | | (30) | |
| | (16) | | | | | | | | | | | | |
| 93662 | ttGCAGGCgGTCGCCaG | DRSYRNT | 0 | RRSDLKR | 1c | ERGTLAR | 0 | DRSALAR | 1c | DRSHLTR | 0 | QSGDLTR | C1 |
| | CGCTCcagcgg | (36) | | (37) | | (38) | | (39) | | (32) | | (27) | |
| | (18) | | | | | | | | | | | | |
| 93752 | agAGAGGGGAGAAGCTT | DRSNLSR | 0 | LKFALAN | 0c | RSDNLST | 0 | RSAALAR | 0c | RSDHLST | 0 | QSAHRIT | C1 |
| | GACcgggtggt | (40) | | (41) | | (42) | | (43) | | (44) | | (45) | |
| | (19) | | | | | | | | | | | | |
| 93548 | tgGCCGTGGATGGCAGG | RSDDLSK | 0 | RSDHRTN | 0c | DRSHLTR | 0 | TSANLSR | 0c | RSDSLSR | 0 | DRSVRTK | C1 |
| | TCGtgcaaccc | (46) | | (47) | | (32) | | (48) | | (49) | | (50) | |
| | (20) | | | | | | | | | | | | |
| 93845 | taTTTGTGTTTGTGtGC | YKHVLSD | 0 | TSGSLTR | 1c | RSDSLLR | 0 | NYASRTW | 0c | RSDSLLR | 0 | NYASRTW | C1 |
| | TCTTaaggggt | (51) | | (52) | | (53) | | (54) | | (53) | | (54) | |
| | (21) | | | | | | | | | | | | |
| 93587 | gtGCAGAGGCTACAGCG | RSDSLSQ | 0 | RKADRTR | 0c | QSGDLTR | 0 | LKDTLRR | 0c | RSANLAR | 0 | QSSDLRR | C1 |
| | TCGccaccacc | (55) | | (56) | | (27) | | (57) | | (58) | | (59) | |
| | (22) | | | | | | | | | | | | |
| 93563 | agGGGGTTGCACGACCT | HKTSLKD | | | 0a | QSNHLTE | 0) | QNATRTK | 0c | DRSALSR | 0 | RSDHLSR | C1 |
| | gccatccacgg | (60) | | | | (61) | | (62) | | (63) | | (64) | |
| | (23) | | | | | | | | | | | | |
| 93582 | cgCATGGTGCAGAGGCT | QSGDLTR | 0 | LKDTLRR | 0c | RSANLAR | 0 | QSSDLRR | 0c | LRHHLTR | 0 | LRHNLRA | C1 |
| | ACAgcgtcgcc | (27) | | (57) | | (58) | | (59) | | (65) | | (66) | |
| | (24) | | | | | | | | | | | | |

TABLE 2

| ZFP ID | Amino Acid sequence (helix, ZF linker, & interdomain linker) |
|---|---|
| 93598 | MAPKKKRKVGVPAAMAERPFQCRICMRNFSDRSDLSRHIRTHTGEKPFACDICGRKFAQSGSLTRHTKIHTGSQKPFQCRICMRNFSQS |
| | GDLTRHIRTHTGEKPFACDICGRKFADSSNRAKHTKIHTGSQKPFQCRICMRNFSDRSHLSRHIRTHTGEKPFACDICGRKFATSGHLSR |
| | HTKIHLROKDAARGSGGDAKSLTAWSRTLVTFKDVFVDFTREEWKLLDTAQQIVYRNVMLENYKNLVSLGYQLTKPDVILRLEKGEEP |
| | WLVEREIHQETHPDSETAFEIKSSVRS (SEQ ID NO: 72) |
| 93600 | MAPKKKRKVGVPAAMAERPFQCRICMRNFSASKTRTNHIRTHTGEKPFACDICGRKFAQSGSLTRHTKIHTGSQKPFQCRICMRNFSQS |
| | GDLTRHIRTHTGEKPFACDICGRKFADSSNRAKHTKIHTGSQKPFQCRICMRNFSDRSHLTRHIRTHTGEKPFACDICGRKFATSGHLSR |
| | HTKIHLROKDAARGSGGDAKSLTAWSRTLVTFKDVFVDFTREEWKLLDTAQQIVYRNVMLENYKNLVSLGYQLTKPDVILRLEKGEEP |
| | WLVEREIHQETHPDSETAFEIKSSVRS (SEQ ID NO: 73) |
| 93591 | MAPKKKRKVGVPAAMAERPFQCRICMRNFSASKTRTNHIRTHTGEKPFACDICGRKFAQSGSLTRHTKIHTGSQKPFQCRICMRNFSQS |
| | GDLTRHIRTHTGEKPFACDICGRKFADSSNRAKHTKIHTGEKPFQCRICMRKFADSSHRTRHTKIHLROKDAARGSGGDAKSLTAWSRT |
| | LVTFKDVFVDFTREEWKLLDTAQQIVYRNVMLENYKNLVSLGYQLTKPDVILRLEKGEEPWLVEREIHQETHPDSETAFEIKSSVRS |
| | (SEQ ID NO: 74) |
| 93590 | MAPKKKRKVGVPAAMAERPFQCRICMRNFSASKTRTNHIRTHTGEKPFACDICGRKFAQSGSLTRHTKIHTGSQKPFQCRICMRNFSQS |
| | GDLTRHIRTHTGEKPFACDICGRKFADRSNLTRHTKIHTGEKPFQCRICMRKFADRSHLARHTKIHLROKDAARGSGGDAKSLTAWSR |
| | TLVTFKDVFVDFTREEWKLLDTAQQIVYRNVMLENYKNLVSLGYQLTKPDVILRLEKGEEPWLVEREIHQETHPDSETAFEIKSSVRS |
| | (SEQ ID NO: 76) |
| 93599 | MAPKKKRKVGVPAAMAERPFQCRICMRNFSASKTRTNHIRTHTGEKPFACDICGRKFAQSGSLTRHTKIHTGSQKPFQCRICMRNFSQS |
| | GDLTRHIRTHTGEKPFACDICGRKFADRSNLTRHTKIHTGSQKPFQCRICMRNFSDRSHLSRHIRTHTGEKPFACDICGRKFATSGHLSR |
| | HTKIHLROKDAARGSGGDAKSLTAWSRTLVTFKDVFVDFTREEWKLLDTAQQIVYRNVMLENYKNLVSLGYQLTKPDVILRLEKGEEP |
| | WLVEREIHQETHPDSETAFEIKSSVRS (SEQ ID NO: 77) |
| 93662 | MAPKKKRKVGVPAAMAERPFQCRICMRNFSDRSYRNTHIRTHTGEKPFACDICGRKFARRSDLKRHTKIHTHPRAPIPKPFQCRICMRN |
| | FSERGTLARHIRTHTGEKPFACDICGRKFADRSALARHTKIHTHPRAPIPKPFQCRICMRNFSDRSHLTRHIRTHTGEKPFACDICGRKFA |
| | QSGDLTRHTKIHLRQKDAARGSGGDAKSLTAWSRTLVTFKDVFVDFTREEWKLLDTAQQIVYRNVMLENYKNLVSLGYQLTKPDVIL |
| | RLEKGEEPWLVEREIHQETHPDSETAFEIKSSVRS (SEQ ID NO: 78) |
| 93752 | MAPKKKRKVGVPAAMAERPFQCRICMRNFSDRSNLSRHIRTHTGEKPFACDICGRKFALKFALANHTKIHTGSQKPFQCRICMRNFSRS |
| | DNLSTHIRTHTGEKPFACDICGRKFARSAALARHTKIHTGSQKPFQCRICMRNFSRSDHLSTHIRTHTGEKPFACDICGRKFAQSAHRIT |
| | HTKIHLROKDAARGSGGDAKSLTAWSRTLVTFKDVFVDFTREEWKLLDTAQQIVYRNVMLENYKNLVSLGYQLTKPDVILRLEKGEEP |
| | WLVEREIHQETHPDSETAFEIKSSVRS (SEQ ID NO: 79) |
| 93548 | MAPKKKRKVGVPAAMAERPFQCRICMRNFSRSDDLSKHIRTHTGEKPFACDICGRKFARSDHRTNHTKIHTGSQKPFQCRICMRNFSD |
| | RSHLTRHIRTHTGEKPFACDICGRKFATSANLSRHTKIHTGSQKPFQCRICMRNFSRSDSLSRHIRTHTGEKPFACDICGRKFADRSVRT |
| | KHTKIHLROKDAARGSGGDAKSLTAWSRTLVTFKDVFVDFTREEWKLLDTAQQIVYRNVMLENYKNLVSLGYQLTKPDVILRLEKGE |
| | EPWLVEREIHQETHPDSETAFEIKSSVRS (SEQ ID NO: 80) |
| 93845 | MAPKKKRKVGVPAAMAERPFQCRICMRNFSYKHVLSDHIRTHTGEKPFACDICGRKFATSGSLTRHTKIHTHPRAPIPKPFQCRICMRN |
| | FSRSDSLLRHIRTHTGEKPFACDICGRKFANYASRTWHTKIHTGSQKPFQCRICMRNFSRSDSLLRHIRTHTGEKPFACDICGRKFANYA |
| | SRTWHTKIHLROKDAARGSGGDAKSLTAWSRTLVTFKDVFVDFTREEWKLLDTAQQIVYRNVMLENYKNLVSLGYQLTKPDVILRLE |
| | KGEEPWLVEREIHQETHPDSETAFEIKSSVRS (SEQ ID NO: 81) |
| 93587 | MAPKKKRKVGVPAAMAERPFQCRICMRNFSRSDSLSQHIRTHTGEKPFACDICGRKFARKADRTRHTKIHTGSQKPFQCRICMRNFSQS |
| | GDLTRHIRTHTGEKPFACDICGRKFALKDTLRRHTKIHTGSQKPFQCRICMRNFSRSANLARHIRTHTGEKPFACDICGRKFAQSSDLR |
| | RHTKIHLROKDAARGSGGDAKSLTAWSRTLVTFKDVFVDFTREEWKLLDTAQQIVYRNVMLENYKNLVSLGYQLTKPDVILRLEKGEE |
| | PWLVEREIHQETHPDSETAFEIKSSVRS (SEQ ID NO: 82) |
| 93563 | MAPKKKRKVGVPAAMAERPFQCRICMRKFAHKTSLKDHTKIHTGEKPFQCRICMRNFSQSNHLTEHIRTHTGEKPFACDICGRKFAQN |
| | ATRTKHTKIHTGSQKPFQCRICMRNFSDRSALSRHIRTHTGEKPFACDICGRKFARSDHLSRHTKIHLRQKDAARGSGGDAKSLTAWSR |
| | TLVTFKDVFVDFTREEWKLLDTAQQIVYRNVMLENYKNLVSLGYQLTKPDVILRLEKGEEPWLVEREIHQETHPDSETAFEIKSSVRS |
| | (SEQ ID NO: 83) |
| 93582 | MAPKKKRKVGVPAAMAERPFQCRICMRNFSQSGDLTRHIRTHTGEKPFACDICGRKFALKDTLRRHTKIHTGSQKPFQCRICMRNFSR |
| | SANLARHIRTHTGEKPFACDICGRKFAQSSDLRRHTKIHTGSQKPFQCRICMRNFSLRHHLTRHIRTHTGEKPFACDICGRKFALRHNL |
| | RAHTKIHLROKDAARGSGGDAKSLTAWSRTLVTFKDVFVDFTREEWKLLDTAQQIVYRNVMLENYKNLVSLGYQLTKPDVILRLEKG |
| | EEPWLVEREIHQETHPDSETAFEIKSSVRS (SEQ ID NO: 84) |

TABLE 3

| | Target Sequence | DNA-Recognition Helix Amino Acid | | | | | | | | | | | |
|---|---|---|---|---|---|---|---|---|---|---|---|---|---|
| | (capital | Sequence Within Each Finger | | | | | | | | | | | |
| ZFP UD | letters) | F1 | L1 | F2 | F2 | F3 | L3 | F4 | L4 | F5 | L5 | F6 | L6 |
| 96963 | gtGGCGACGCTGTA | \hat{ }ASKTRTN | 0 | QSGSLTR | 0c | \hat{ }QSGDLTR | 0 | DRSNLTR | 0a | DRSHLAR | | | C1 |
| | GCCtctgcaccatg | (67) | | (26) | | (68) | | (34) | | (35) | | | |
| | (17) | | | | | | | | | | | | |
| 96973 | gtGGTGGCGACGCT | \hat{ }DRSDLSR | 0 | QSGSLTR | 0c | QSGDLTR | 0 | DSSNRAK | 0c | DRSHLSR | 0 | TSGH | C1 |
| | GTAGCCtctgcacc | (69) | | (26) | | (27) | | (28) | | (29) | | LSR | |
| | (16) | | | | | | | | | | | (30) | |
| 97028 | ttGCAGGCgGTCGC | \hat{ }DRSYRNT | 0 | RRSDLKR | 0c | \hat{ }ERGTLAR | 0 | DRSALAR | 1c | DRSHLTR | 0 | QSGD | C1 |
| | CaGCGCTCcagcgg | (70) | | (37) | | (71) | | (39) | | (32) | | LTR | |
| | (18) | | | | | | | | | | | (27) | |
| 96967 | gtGGCGACGCTGTA | \hat{ }ASKTRTN | 0 | QSGSLTR | 0c | QSGDLTR | 0 | DSSNRAK | 0a | DSSHRTR | | | C1 |
| | GCCtctgcaccatg | (67) | | (26) | | (27) | | (28) | | (33) | | | |
| | (17) | | | | | | | | | | | | |
| 96968 | gtGGCGACGCTGTA | ASKTRTN | 0 | QSGSLTR | 0c | \hat{ }QSGDLTR | 0 | DSSNRAK | 0a | DSSHRTR | | | C1 |
| | GCCtctgcaccatg | (31) | | (26) | | (68) | | (28) | | (33) | | | |
| | (17) | | | | | | | | | | | | |
| 96980 | gtGGTGGCGACGCT | \hat{ }ASKTRTN | 0 | QSGSLTR | 0c | \hat{ }QSGDLTR | 0 | DRSNLTR | 0c | DRSHLSR | 0 | TSGH | C1 |
| | GTAGCCtctgcacc | (67) | | (26) | | (68) | | (34) | | (29) | | LSR | |
| | (16) | | | | | | | | | | | (30) | |
| 96986 | gtGGTGGCGACGCT | \hat{ }ASKTRTN | 0 | QSGSLTR | 0c | \hat{ }QSGDLTR | 0 | DSSNRAK | 0c | DRSHLTR | 0 | TSGH | C1 |
| | GTAGCCtctgcacc | (67) | | (26) | | (68) | | (28) | | (32) | | LSR | |
| | (16) | | | | | | | | | | | (30) | |

TABLE 4

| ZFP ID | Amino Acid sequence (helix, ZF linker, & interdomain linker) |
|---|---|
| 96963 | MAPKKKRKVGVPAAMAERPFQCRICMQNFSASKTRTNHIRTHTGEKPFACDICGRKFAQSGSLTRHTKIHTGSQKPFQCRICMQNFSQ |
| | SGDLTRHIRTHTGEKPFACDICGRKFADRSNLTRHTKIHTGEKPFQCRICMRKFADRSHLARHTKIHLROKDAARGSGGDAKSLTAWS |
| | RTLVTFKDVFVDFTREEWKLLDTAQQIVYRNVMLENYKNLVSLGYQLTKPDVILRLEKGEEPWLVEREIHQETHPDSETAFEIKSSVRS |
| | (SEQ ID NO: 85) |
| 96973 | MAPKKKRKVGVPAAMAERPFQCRICMQNFSDRSDLSRHIRTHTGEKPFACDICGRKFAQSGSLTRHTKIHTGSQKPFQCRICMRNFSQ |
| | SGDLTRHIRTHTGEKPFACDICGRKFADSSNRAKHTKIHTGSQKPFQCRICMRNFSDRSHLSRHIRTHTGEKPFACDICGRKFATSGHLS |
| | RHTKIHLROKDAARGSGGDAKSLTAWSRTLVTFKDVFVDFTREEWKLLDTAQQIVYRNVMLENYKNLVSLGYQLTKPDVILRLEKGE |
| | EPWLVEREIHQETHPDSETAFEIKSSVRS (SEQ ID NO: 86) |
| 97028 | MAPKKKRKVGVPAAMAERPFQCRICMQNFSDRSYRNTHIRTHTGEKPFACDICGRKFARRSDLKRHTKIHTHPRAPIPKPFQCRICMQ |
| | NFSERGTLARHIRTHTGEKPFACDICGRKFADRSALARHTKIHTHPRAPIPKPFQCRICMRNFSDRSHLTRHIRTHTGEKPFACDICGRK |
| | FAQSGDLTRHTKIHLRQKDAARGSGGDAKSLTAWSRTLVTFKDVFVDFTREEWKLLDTAQQIVYRNVMLENYKNLVSLGYQLTKPD |
| | VILRLEKGEEPWLVEREIHQETHPDSETAFEIKSSVRS (SEQ ID NO: 87) |
| 96967 | MAPKKKRKVGVPAAMAERPFQCRICMQNFSASKTRTNHIRTHTGEKPFACDICGRKFAQSGSLTRHTKIHTGSQKPFQCRICMRNFSQ |
| | SGDLTRHIRTHTGEKPFACDICGRKFADSSNRAKHTKIHTGEKPFQCRICMRKFADSSHRTRHTKIHLRQKDAARGSGGDAKSLTAWS |
| | RTLVTFKDVFVDFTREEWKLLDTAQQIVYRNVMLENYKNLVSLGYQLTKPDVILRLEKGEEPWLVEREIHQETHPDSETAFEIKSSVRS |
| | (SEQ ID NO: 88) |
| 96968 | MAPKKKRKVGVPAAMAERPFQCRICMRNFSASKTRTNHIRTHTGEKPFACDICGRKFAQSGSLTRHTKIHTGSQKPFQCRICMQNFSQ |
| | SGDLTRHIRTHTGEKPFACDICGRKFADSSNRAKHTKIHTGEKPFQCRICMRKFADSSHRTRHTKIHLRQKDAARGSGGDAKSLTAWS |
| | RTLVTFKDVFVDFTREEWKLLDTAQQIVYRNVMLENYKNLVSLGYQLTKPDVILRLEKGEEPWLVEREIHQETHPDSETAFEIKSSVRS |
| | (SEQ ID NO: 89) |
| 96980 | MAPKKKRKVGVPAAMAERPFQCRICMQNFSASKTRTNHIRTHTGEKPFACDICGRKFAQSGSLTRHTKIHTGSQKPFQCRICMQNFSQ |
| | SGDLTRHIRTHTGEKPFACDICGRKFADRSNLTRHTKIHTGSQKPFQCRICMRNFSDRSHLSRHIRTHTGEKPFACDICGRKFATSGHLS |
| | RHTKIHLRQKDAARGSGGDAKSLTAWSRTLVTFKDVFVDFTREEWKLLDTAQQIVYRNVMLENYKNLVSLGYQLTKPDVILRLEKGE |
| | EPWLVEREIHQETHPDSETAFEIKSSVRS (SEQ ID NO: 90) |
| 96986 | MAPKKKRKVGVPAAMAERPFQCRICMQNFSASKTRTNHIRTHTGEKPFACDICGRKFAQSGSLTRHTKIHTGSQKPFQCRICMQNFSQ |
| | SGDLTRHIRTHTGEKPFACDICGRKFADSSNRAKHTKIHTGSQKPFQCRICMRNFSDRSHLTRHIRTHTGEKPFACDICGRKFATSGHLS |
| | RHTKIHLROKDAARGSGGDAKSLTAWSRTLVTFKDVFVDFTREEWKLLDTAQQIVYRNVMLENYKNLVSLGYQLTKPDVILRLEKGE |
| | EPWLVEREIHQETHPDSETAFEIKSSVRS (SEQ ID NO: 91) |

The ZFP domain of the present engineered ZFP fusions may include at least one (e.g., one, two, three, four, five, six, seven, eight, nine, ten, eleven, twelve, thirteen, or more) zinc finger(s). A ZFP domain having one finger typically recognizes a target site that includes 3 or 4 nucleotides. A ZFP domain having two fingers typically recognizes a target site that includes 6 or 8 nucleotides. A ZFP domain having three fingers typically recognizes a target site that includes 9 or 12 nucleotides. A ZFP domain having four fingers typically recognizes a target site that includes 12 to 15 nucleotides. A ZFP domain having five fingers typically recognizes a target site that includes 15 to 18 nucleotides. A ZFP domain having six fingers can recognize target sites that include 18 to 21 nucleotides.

In some embodiments, the present engineered ZFPs comprise a DNA-binding recognition helix sequence having at least 4 of the amino acids of any recognition helix as shown in Table 1 or Table 3. In other embodiments, the present engineered ZFPs comprise a DNA-binding recognition helix sequence shown in Table 1 or Table 3. For example, an engineered ZFP may comprise the sequence of F1, F2, F3, F4, F5, or F6 as shown in Table 1 or Table 3.

In some embodiments, the present engineered ZFPs comprise two adjacent DNA-binding recognition helix sequences shown in a single row of Table 1 or Table 3. For example, an engineered ZFP may comprise the sequences of F1-F2, F2-F3, F3-F4, F4-F5, or F5-F6 as shown in a single row of Table 1 or Table 3. In some embodiments, the engineered ZFP may comprises at least 4 amino acids of F1 and at least 4 amino acids of F2, at least 4 amino acids of F2 and at least 4 amino acids of F3, at least 4 amino acids of F3 and at least 4 amino acids of F4, at least 4 amino acids of F4 and at least 4 amino acids of F5, or at least 4 amino acids of F5 and at least 4 amino acids of F6.

In some embodiments, the present engineered ZFPs comprise the DNA-binding recognition helix sequences shown in a single row of Table 1 or Table 3. For example, an engineered ZFP may comprise the sequences of F1, F2, F3, F4, F5, and F6 (e.g., F1-F4, F1-F5, or F1-F6) as shown in a single row of Table 1 or Table 3. In some embodiments, the engineered ZFP comprises at least 4 amino acids of each of F1-F4, at least 4 amino acids of each of F1-F5, or at least 4 amino acids of each of F1-F6 as shown in a single row of Table 1 or Table 3.

In some embodiments, an engineered ZFP described herein comprises the recognition helix and backbone portions of a sequence shown in a single row of Table 2 or Table 4. In some embodiments, an engineered ZFP described herein comprises the recognition helix and backbone portions of a sequence shown in a single row of Table 2 or Table 4 as the sequence would appear following post-translational modification. For example, post-translational modification may remove the initiator methionine residue from a sequence as shown in Table 2 or Table 4.

The target specificity of the ZFP domain may be improved by mutations to the ZFP backbone sequence as described in, e.g., U.S. Pat. No. 10,975,393. The backbone mutations include those made to residues that can interact non-specifically with phosphates on the DNA backbone but are not involved in nucleotide target specificity. In some embodiments, these mutations comprise mutating a cationic amino acid residue to a neutral or anionic amino acid residue. In some embodiments, these mutations comprise mutating a polar amino acid residue to a neutral or non-polar amino acid residue. In further embodiments, mutations are made at positions (−4), (−5), (−9) and/or (−14) relative to the DNA-binding helix. In some embodiments, a zinc finger may comprise one or more mutations at positions (−4), (−5), (−9) and/or (−14). In further embodiments, one or more zinc fingers in a multi-finger ZFP domain may comprise mutations at positions (−4), (−5), (−9) and/or (−14). In some embodiments, the amino acids at positions (−4), (−5), (−9) and/or (−14) (e.g., an arginine (R) or lysine (K)) are mutated to an alanine (A), leucine (L), serine(S), aspartate (D), glutamate (E), tyrosine (Y), and/or glutamine (Q). In some embodiments, the R residue at position (−5) is mutated to Q. The symbol “\hat{ }” in Table 3 indicates that the arginine (R) residue at the 4th position upstream of the 1st amino acid in the indicated recognition helix is changed to glutamine (Q). In each recognition helix sequence, the positions of the seven DNA-binding amino acids are numbered −1, +1, +2, +3, +4, +5, and +6. Thus, the position for the R-to-Q substitution is numbered as (−5).

In some embodiments, the present engineered ZFPs comprise a DNA-binding recognition helix sequence and associated backbone mutation as shown in Table 3. In some embodiments, the present engineered ZFPs comprise the DNA-binding recognition helix sequences and associated backbone mutations as shown in a single row of Table 3.

In some embodiments, an engineered ZFP described herein comprises the recognition helix and backbone portions of a sequence shown in a single row of Table 4. In some embodiments, an engineered ZFP described herein comprises the recognition helix and backbone portions of a sequence shown in a single row of Table 4 as the sequence would appear following post-translational modification. For example, post-translational modification may remove the initiator methionine residue from a sequence as shown in Table 4.

In some embodiments, the present ZFP protein comprises the recognition helix sequences and/or the amino acid sequence shown in a single row in Tables 1~4 for each of ZFP ID's 93598, 93600, 93591, 93590, 93599, 93662, 93752, 93548, 93845, 93587, 93563, 93582, 96963, 96973, 97028, 96967, 96968, 96980, or 96986.

### III. Zinc Finger Protein Transcription Factors

The ZFP domains described herein may be fused to a transcription factor. In some embodiments, the present fusion proteins contain a DNA-binding zinc finger protein (ZFP) domain and a transcription factor domain (i.e., ZFP-TF). In some embodiments, the transcription factor may be a transcription repressor domain, wherein the ZFP and repressor domains may be associated with each other by a direct peptidyl linkage or a peptide linker, or by dimerization (e.g., through a leucine zipper, a STAT protein N terminal domain, or an FK506 binding protein). As used herein, a “fusion protein” refers to a polypeptide with covalently linked domains as well as a complex of polypeptides associated with each other through non-covalent bonds. The transcription repressor domain can be associated with the ZFP domain at any suitable position, including the C- or N-terminus of the ZFP domain.

In some embodiments, the present ZFP-TFs bind to their target with a K_{D} of less than about 25 nM and repress transcription of a human SCN9A gene by 20% or more (e.g., by 30%, 40%, 50%, 60%, 70%, 80%, 90%, 95%, 99%, or more). In some embodiments, two or more of the present ZFP-TFs are expressed in a cell to synergistically modulate SCN9A expression in the cell (see, e.g., U.S. Patent Application Publication Nos. 2020/0101133 and 2020/0109406). Such synergistic ZFP-TFs may be linked by a 2A linker peptide, e.g., T2A GSGEGRGSLLTCGDVEENPGP (SEQ ID NO:110). Thus, two or more of the present ZFP-TFs may be used concurrently in a patient, where the ZFP-TFs bind to different target regions in the SCN9A gene, so as to achieve optimal repression of SCN9A expression.

In some embodiments, the present ZFP-TFs comprise one or more zinc finger domains. The domains may be linked together via an extendable flexible linker such that, for example, one domain comprises one or more (e.g., 4, 5, or 6) zinc fingers and another domain comprises an additional one or more (e.g., 4, 5, or 6) zinc fingers. In some embodiments, the linker is a standard inter-finger linker such that the finger array comprises one DNA-binding domain comprising 8, 9, 10, 11 or 12 or more fingers. In other embodiments, the linker is an atypical linker such as a flexible linker. For example, two ZFP domains may be linked to a transcription repressor TF in the configuration (from N terminus to C terminus) ZFP-ZFP-TF, TF-ZFP-ZFP, ZFP-TF-ZFP, or ZFP-TF-ZFP-TF (two ZFP-TF fusion proteins are fused together via a linker).

In some embodiments, the ZFP-TFs are “two-handed,” i.e., they contain two zinc finger clusters (two ZFP domains) separated by intervening amino acids so that the two ZFP domains bind to two discontinuous target sites. An example of a two-handed type of zinc finger binding protein is SIP1, where a cluster of four zinc fingers is located at the amino terminus of the protein and a cluster of three fingers is located at the carboxyl terminus (see Remacle et al., EMBO J. (1999) 18(18): 5073-84). Each cluster of zinc fingers in these proteins is able to bind to a unique target sequence and the spacing between the two target sequences can comprise many nucleotides.

In some embodiments, an engineered ZFP-TF described herein binds to a target site as shown in a single row of Table 1 or Table 3, preferably with no or little detectable off-target binding or activity. Off-target binding may be determined, for example, by measuring the activity of ZFP-TFs at off-target genes. In some embodiments, an engineered ZFP-TF described herein comprises a DNA-binding recognition helix sequence shown in Table 1 or Table 3. In some embodiments, an engineered ZFP-TF described herein comprises two adjacent DNA-binding recognition helix sequences shown in a single row of Table 1 or Table 3. In some embodiments, an engineered ZFP-TF described herein comprises the DNA-binding recognition helix sequences shown in a single row of Table 1 or Table 3.

### A. Transcription Repressor Domains

The present ZFP-TFs comprise an engineered ZFP domain as described herein and one or more transcription repressor domains that dampen the transcription activity of the SCN9A gene. One or more engineered ZFP domains and one or more transcription repressor domains may be joined by a flexible linker. Non-limiting examples of transcription repressor domains are the KRAB domain of KOX1 or ZIM3 (or any other KRAB domain containing protein; see, e.g., Alerasool et al., Nature Methods (2020) 17:1093-6), KAP-1, MAD, FKHR, EGR-1, ERD, SID, TGF-beta-inducible early gene (TIEG), v-ERB-A, MBD2, MBD3, TRa, histone methyltransferase, histone deacetylase (HDAC), nuclear hormone receptor (e.g., estrogen receptor or thyroid hormone receptor), members of the DNMT family (e.g., DNMT1, DNMT3A, DNMT3B), Rb, and MeCP2. See, e.g., Bird et al., Cell (1999) 99:451-4; Tyler et al., Cell (1999) 99:443-6; Knoepfler et al., Cell (1999) 99:447-50; and Robertson et al., Nature Genet. (2000) 25:338-42. Additional exemplary repression domains include, but are not limited to, ROM2 and AtHD2A. See, e.g., Chem et al., Plant Cell (1996) 8:305-21; and Wu et al., Plant J. (2000) 22:19-27.

In some embodiments, the transcription repressor domain comprises a Kruppel-associated box (KRAB) domain that is from a KOX1 protein. In some embodiments, the KOX1 protein is human. In some embodiments, the transcription repressor domain comprises a sequence from the Kruppel-associated box (KRAB) domain of the human zinc finger protein 10/KOX1 (ZNF10/KOX1) (e.g., GenBank No. NM_015394.4). An exemplary KRAB domain sequence is:

(SEQ ID NO: 2)
DAKSLTAWSR TLVTFKDVFV DFTREEWKLL DTAQQIVYRN
VMLENYKNLV SLGYQLTKPD VILRLEKGEE PWLVEREIHQ
ETHPDSETAF EIKSSV

Variants of this KRAB sequence may also be used so long as they have the same or similar transcription repressor function.

### B. Peptide Linkers

The ZFP domain and the transcription repressor domain of the present ZFP-TFs and/or the zinc fingers within the ZFP domains may be linked through a peptide linker, e.g., a noncleavable peptide linker of about 5 to 50 amino acids (e.g., 5, 6, 7, 8, 9, 10, 11, 12, 13, 14, 15, 16, 17, 18, 19, 20 or more amino acids). Preferred linkers are typically flexible amino acid subsequences that are synthesized as a recombinant fusion protein. In some embodiments, zinc fingers are linked such that there is no gap between the linked module target subsites in the target nucleic acid molecule. In other embodiments, zinc fingers are linked by linkers designed to allow the linked modules to bind to target sites with 1, 2 or 3 base pair gaps between the linked module target subsites in the target nucleic acid molecule. See, e.g., U.S. Pat. No. 8,772,453.

In some embodiments, the peptide linker is 3 to 20 amino acid residues in length and is rich in G and/or S. Non-limiting examples of such linkers are G₄S-type linkers, i.e., linkers containing one or more (e.g., 2, 3, or 4) GGGGS (SEQ ID NO: 15) motifs, or variations of the motif (such as ones that have 1, 2, or 3 amino acid insertions, deletions, and substitutions from the motif).

Linker design methods and illustrative linkers that may be used to link the ZFP domain and the transcription repressor domain of the present ZFP-TFs and/or the zinc fingers within the ZFP domains are described in U.S. Pat. Nos. 6,479,626; 7,851,216; 8,772,453; 9,394,531; 9,567,609; and 10,724,020; and PCT Publication Nos. WO 1999/045132; WO 2001/053480; WO 2009/154686; WO 2011/139349; WO 2015/031619; and WO 2017/136049. The proteins described herein may include any combination of suitable linkers.

Non-limiting examples of linkers are DGGGS (SEQ ID NO: 3), TGEKP (SEQ ID NO: 4), LRQKDGERP (SEQ ID NO: 5), GGRR (SEQ ID NO: 6), GGRRGGGS (SEQ ID NO: 7), LRQRDGERP (SEQ ID NO: 8), LRQKDGGGSERP (SEQ ID NO: 9), LRQKD (G₃S)₂ ERP (SEQ ID NO: 10), TGSQKP (SEQ ID NO: 11), LRQKDAARGS (SEQ ID NO: 13), and LRQKDAARGSGG (SEQ ID NO: 14). Additional illustrative linkers for linking zinc fingers and/or for linking domains are listed in Table 5. The finger-finger linkers listed in Table 5 include portions of backbone sequence, e.g., FQ or FA.

Table 5 shows illustrative alternate peptide linkers that may be used to link zinc finger amino acid sequences and/or ZFP and functional domain sequences as shown in Table 1 or Table 3.

TABLE 5

| | | Exemplary Linker | | |
|---|---|---|---|---|
| Linker Position | Linker Category | Linker Code | Peptide sequence | SEQ ID NO: |
| Finger-Finger | No base skipping | 0a | TGEKPFQ | 93 |
| | | 0b | TGGQRPFQ | 94 |
| | | 0c | TGSQKPFQ | 95 |
| | | 0d | TGSQRPFQ | 96 |
| | | 0f | TGEKPFA | 97 |
| | 1 base skipping | 1a | TGGGGSQRPFQ | 98 |
| | | 1b | TGGGGSQKPFQ | 99 |
| | | 1c | THPRAPIPKPFQ | 100 |
| | | 1d | TPNRRPAPKPFQ | 101 |
| | | 1e | TVPRPTPPKPFQ | 102 |
| | | 1f | TYPRPIAAKPFQ | 103 |
| | 2 base skipping | 2a | TGGGGSGGSQRPFQ | 104 |
| | | 2b | TGGGGSGGSQKPFQ | 105 |
| | | 2d | TLAPRPYRPPKPFQ | 106 |
| | | 2e | TPGGKSSRTDRNKPFQ | 107 |
| | | 2f | TPNPHRRTDPSHKPFQ | 108 |
| ZFP-functional | ZFP-TF | C0 | LRGSGG | 109 |
| domain | | C1 | LRQKDAARGS | 13 |
| (interdomain) | | Clk | LRQKDAARGSGG | 14 |

In some embodiments, an engineered ZFP, e.g., as described herein, comprises two adjacent DNA-binding recognition helix sequences linked as shown in a single row of Table 1 or Table 3. For example, an engineered ZFP may comprise the sequences of F1-F2, F2-F3, F3-F4, F4-F5, or F5-F6 as shown in a single row of Table 1 or Table 3. In other embodiments, one or more different linkers may be used from the same linker category.

In some embodiments, an engineered ZFP, e.g., as described herein, comprises the DNA-binding recognition helix sequences linked as shown in a single row of Table 1 or Table 3. For example, an engineered ZFP may comprise the linked sequences of F1-F4, F1-F5, or F1-F6 as shown in a single row of Table 1 or Table 3. In other embodiments, one or more different linkers may be used from the same linker category.

In some embodiments, an engineered ZFP, e.g., as described herein, comprises the recognition helix and linker portions of a sequence as shown in a single row of Table 1 or Table 3. In other embodiments, one or more different linkers may be used from the same linker category. In some embodiments, an engineered ZFP described herein comprises the recognition helix, backbone, and linker portions of a sequence as shown in a single row of Table 2 or Table 4. In other embodiments, one or more different linkers may be used from the same linker category. In some embodiments, an engineered ZFP-TF described herein comprises an amino acid sequence as shown in a single row of Table 2 or Table 4. In some embodiments, an engineered ZFP or ZFP-TF described herein comprises the recognition helix, backbone, and linker portions of a sequence shown in a single row of Table 2 or Table 4 as the sequence would appear following post-translational modification. In some embodiments, an engineered ZFP or ZFP-TF described herein comprises an amino acid sequence as shown in a single row of Table 2 or Table 4 as the sequence would appear following post-translational modification. For example, post-translational modification may remove the initiator methionine residue from a sequence as shown in Table 2 or Table 4.

### IV. Expression of the ZFP-TFs

A ZFP-TF of the present disclosure may be introduced to a patient through a nucleic acid molecule encoding it. The nucleic acid molecule may be an RNA or cDNA molecule. The nucleic acid may be introduced into the brain, spinal cord, or cerebrospinal fluid (CSF) of the patient through injection of a composition comprising a lipid: nucleic acid complex (e.g., a liposome). Alternatively, the ZFP-TF may be introduced to the patient through a nucleic acid expression vector comprising a sequence encoding the ZFP-TF. The expression vectors may include expression control sequences such as promoters, enhancers, transcription signal sequences, and transcription termination sequences that allow expression of the coding sequence for the ZFP-TFs in the cells of the nervous system. In some embodiments, the expression vector remains present in the cell as a stable episome. In other embodiments, the expression vector is integrated into the genome of the cell.

In some embodiments, the promoter to express the ZFP-TFs described herein comprises a constitutive promoter. In some embodiments, the constitutive promoter is constitutively active in neurons. In some embodiments, the promoter comprises an inducible promoter. In some embodiments, the inducible promoter is inducible in neurons.

In some embodiments, the promoter on the vector for directing the ZFP-TF expression in the DRG is a constitutive active promoter or an inducible promoter. Suitable promoters include, without limitation, a Rous sarcoma virus (RSV) long terminal repeat (LTR) promoter (optionally with an RSV enhancer), a cytomegalovirus (CMV) promoter (optionally with a CMV enhancer), a CMV immediate early promoter, a simian virus 40 (SV40) promoter, a dihydrofolate reductase (DHFR) promoter, a β-actin promoter, a phosphoglycerate kinase (PGK) promoter, an EF1a promoter, a Moloney murine leukemia virus (MoMLV) LTR, a creatine kinase-based (CK6) promoter, a transthyretin promoter (TTR), a thymidine kinase (TK) promoter, a tetracycline responsive promoter (TRE), a hepatitis B Virus (HBV) promoter, a human al-antitrypsin (hAAT) promoter, chimeric liver-specific promoters (LSPs), an E2 factor (E2F) promoter, the human telomerase reverse transcriptase (hTERT) promoter, a CMV enhancer/chicken β-actin/rabbit β-globin promoter (CAG promoter; Niwa et al., Gene (1991) 108 (2): 193-9), and an RU-486-responsive promoter. Neuron-specific promoters such as a synapsin I promoter, a calcium/calmodulin-dependent protein kinase II (CamKII) promoter, a methyl CpG-binding protein 2 (MeCP2) promoter, a choline acetyltransferase (ChAT) promoter, a Calbindin (Calb) promoter, a CAMKII promoter, a PrP promoter, a GFAP promoter, or an engineered or natural promoter that restricts expression to neuron and glial cells may also be used. In addition, the promoter may include one or more self-regulating elements whereby the ZFP-TF can bind to and repress its own expression level to a preset threshold. See U.S. Pat. No. 9,624,498.

Any method of introducing the nucleotide sequence into a cell may be employed, including but not limited to, electroporation, calcium phosphate precipitation, microinjection, cationic or anionic liposomes, liposomes in combination with a nuclear localization signal, naturally occurring liposomes (e.g., exosomes), or viral transduction.

For in vivo delivery of an expression vector, viral transduction may be used. A variety of viral vectors known in the art may be adapted by one of skill in the art for use in the present disclosure, for example, vaccinia vectors, adenoviral vectors, lentiviral vectors, poxyviral vectors, adeno-associated viral (AAV) vectors, retroviral vectors, herpes simplex virus (HSV) vectors, and hybrid viral vectors. In some embodiments, the viral vector used herein is a recombinant AAV (rAAV) vector. AAV vectors are especially suitable for nervous system gene delivery because they infect both dividing and non-dividing cells, exist as stable episomal structures for long term expression, and have very low immunogenicity (Hadaczek et al., Mol Ther. (2010) 18:1458-61; Zaiss, et al., Gene Ther. (2008) 15:808-16). Any suitable AAV serotype may be used. For example, the AAV may be AAV1, AAV2, AAV3, AAV3B, AAV4, AAV5, AAV6, AAV7, AAV8, AAV8.2, AAV9, AAV.PHP.B, AAV.PHP.eB, or AAVrh10, or of a novel serotype or a pseudotype such as AAV2/8, AAV2/5, AAV2/6, AAV2/9, or AAV2/6/9, or a serotype that is the variant or derivative of one of the AAV serotypes listed herein (i.e., AAV derived from multiple serotypes; for example, the rAAV comprises AAV2 inverted terminal repeats (ITR) in its genome and an AAV8, 5, 6, or 9 capsid). In some embodiments, the expression vector is an AAV viral vector and is introduced to the target human cell by a recombinant AAV virion whose genome comprises the construct, including having the ITR sequences on both ends to allow the production of the AAV virion in a production system such as an insect cell/baculovirus production system or a mammalian cell production system. The AAV may be engineered such that its capsid proteins have reduced immunogenicity or enhanced transduction ability in humans or nonhuman primates. In some embodiments, AAV9 is used. Viral vectors described herein may be produced using methods known in the art. Any suitable permissive or packaging cell type may be employed to produce the viral particles. For example, mammalian (e.g., 293) or insect (e.g., sf9) cells may be used as the packaging cell line.

### V. Pharmaceutical Applications

The present ZFP-TFs can be used for the treatment of peripheral neuropathic pain disorders such as inherited erythromelalgia (IEM), paroxysmal extreme pain disorder (PEPD), small fiber neuropathy, (idiopathic small fiber neuropathy, small fiber neuropathy plus prediabetes, diabetic small fiber neuropathy), large fiber neuropathy, trigeminal neuralgia, post-herpetic neuralgia, and painful diabetic neuropathy. The present disclosure provides a method of treating a neurological disease (e.g., a pain disorder such as an inherited or chronic pain disorder) in a subject such as a human patient in need thereof, comprising introducing to the nervous system of the subject a therapeutically effective amount (e.g., an amount that allows sufficient repression of SCN9A expression to treat the neurological disease) of the ZFP-TF (e.g., an rAAV vector expressing it). The term “treating” encompasses alleviation of symptoms, prevention of onset of symptoms, slowing of disease progression, improvement of quality of life, and increased survival.

The present disclosure provides a pharmaceutical composition comprising a viral vector such as an rAAV whose recombinant genome comprises an expression cassette for the ZFP-TFs. The pharmaceutical composition (e.g., an artificial cerebrospinal fluid or aCSF), may further comprise a pharmaceutically acceptable carrier such as water, saline (e.g., phosphate-buffered saline), dextrose, glycerol, sucrose, lactose, gelatin, dextran, albumin, or pectin. In addition, the composition may contain auxiliary substances, such as, wetting or emulsifying agents, pH-buffering agents, stabilizing agents, or other reagents that enhance the effectiveness of the pharmaceutical composition. The pharmaceutical composition may contain delivery vehicles such as liposomes, nanocapsules, microparticles, microspheres, lipid particles, and vesicles.

The ZFP-TFs of the present disclosure are under the control of a neuronal specific promoter (e.g., a synapsin promoter), therefore the ZFP-TFs will get expressed in any neuronal subtype in the central or peripheral nervous system. Nav1.7 is expressed along the length of DRG neurons, which extend to the periphery and centrally across the blood-brain barrier (BBB) to synapse with spinal cord second-order neurons. The role of Nav1.7 in these first-order nociceptive sensory neurons has been established as having an essential and non-redundant role in transduction and transmission of pain signaling following noxious stimuli. The target cells for this treatment are the nociceptor neurons in the DRG. The ZFP-TFs of the present disclosure repress the expression of Nav1.7 in nociceptor neurons in the DRG, which prevents or reduces the transmission of pain signals to the brain, thereby stopping the brain from perceiving pain.

The DRGs can be reached through intrahippocampal injection, intracerebral injection, intra-cisterna magna (ICM) injection, or more generally through intraparenchymal injection, intracerebroventricular (ICV) injection, intrathecal injection (IT), or intravenous injection (IV). Other routes of administration include, without limitation, intraventricular, intranasal, oral, or intraocular administrations. In some embodiments, the viral vector can reach the DRG following direct administration into the cerebrospinal fluid (CSF), e.g., via intrathecal and/or intracerebral injection, or intra-cisterna magna injection or intracerebroventricular injection. In other embodiments, the viral vectors are delivered directly to the target regions via epidural administration.

By way of example, the pharmaceutical composition may be provided to the patient through intraventricular administration, e.g., into a ventricular region of the forebrain of the patient such as the right lateral ventricle, the left lateral ventricle, the third ventricle, or the fourth ventricle. The pharmaceutical composition may also be provided to the patient through intrathecal (IT) administration, e.g., administration of the composition by injection into the subarachnoid space of the spinal cord. The pharmaceutical composition may also be provided to the patient through intra-cisterna magna (ICM) administration, e.g., administration of the composition by injection into the cerebrospinal fluid (CSF)-filled subarachnoid space between the cerebellum and the dorsal side of the medulla oblongata.

Delivery of rAAVs to a subject may be accomplished, for example, by intravenous administration. In certain instances, it may be desirable to deliver the rAAVs (e.g., 10¹⁰-10¹⁵ Vg) locally to the DRG, spinal cord, cerebrospinal fluid (CSF), neuronal cells, glial cells, meninges, astrocytes, oligodendrocytes, microglia, interstitial spaces, and the like. AAVs may be delivered with a needle, catheter or related device, using neurosurgical techniques known in the art, such as by stereotactic injection (see, e.g., Stein et al., J Vir. (1999) 73:3424-9; Davidson et al., PNAS (2000) 97:3428-32; Davidson et al., Nat Genet. (1993) 3:219-223; and Alisky and Davidson, Hum Gene Ther. (2000) 11:2315-29).

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety, as if each individual reference were specifically and individually indicated to be incorporated by reference in its entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Any molecule or vector disclosed herein can be used in any of the treatment method here, wherein the individual to be treated is as defined anywhere herein. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: Screening of Anti-SCN9A ZFP-TFs

In order to identify human/NHP and mice specific ZFP-TFs that repress the expression of human SCN9A gene or the homologous Sen9a gene in mice, a library of ZFP-TFs were designed targeting human SCN9A or mouse Scn9a gene spanning from 1000 bp upstream to 500 bp downstream of the TSS and screened for SCN9A repression activity. In this study, a KRAB domain sequence (SEQ ID NO: 2) was used as the transcription repressor and fused to the C-terminus of the ZFP domain.

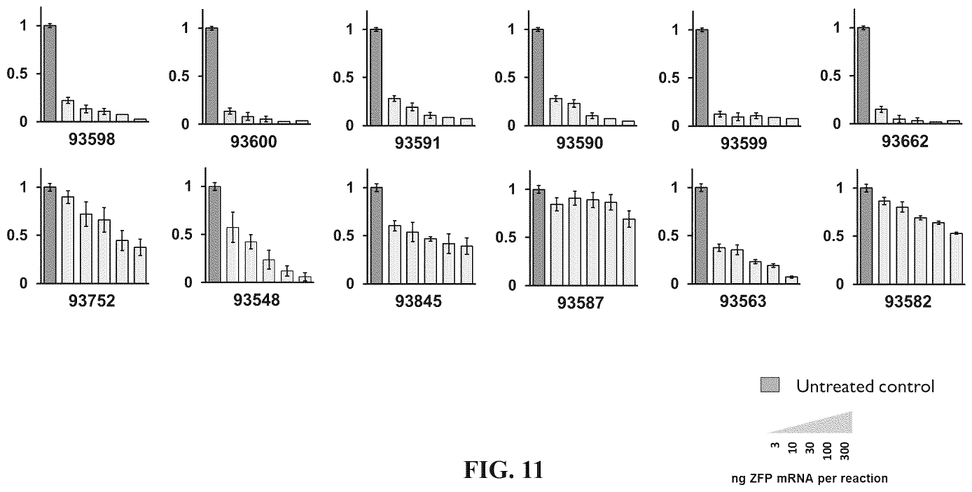

The screening for human ZFP-TFs was performed in the SK-N-MC human neuroepithelial cell line while the screening for mice ZFP-TFs was performed in mice Neuro2A (N2A) neural crest-derived cell lines. Both SK-N-MC and N2A cells express the SCN9A gene at high levels and are thus appropriate for testing of ZFP-TFs that reduce SCN9A expression. Each cell line was cultured in tissue culture flasks until confluency. The cells were plated on 96-well plates and were resuspended in Amaxa® SF solution. The cells were then mixed with ZFP-TF mRNA (3 doses for mice: 30, 100, 300 ng, and 5 doses for human: 3, 10, 30, 100, 300 ng) and transferred to Amaxa® shuttle plate wells. The cells were transfected using the Amaxa® Nucleofector® device (Lonza; program CM-137). Eagle's MEM cell media was added to each well of the plate. The cells were transferred to a 96-well tissue culture plate and incubated at 37° C. for 20 hours. The cells were then lysed and reverse transcription was performed using the C2CT kit following the manufacturer's instructions. TaqMan quantitative polymerase chain reaction (qPCR) was used to measure the expression levels of SCN9A, which were normalized to the geometric mean of the expression levels of the housekeeping genes ATP5B and EIF4A2. A mock transfection and transfection with a ZFP-TF known not to target SCN9A were used as negative controls. The maximum repression achieved was 100%, but we also identified ZFP-TFs that repressed SCN9A to a lesser degree (e.g., about 90%, about 75%, or about 50% at the highest dose or in some instances no repression). FIG. 2 shows the screening data for mice and FIG. 11 shows the screening data for human ZFP-TFs.

### Example 2: AAV Production for Exemplary ZFP-TFs

Recombinant adeno-associated virus (rAAV) vectors were generated by the triple transfection method. Briefly, HEK293 cells were plated in ten-layer CellSTACK® chambers (Corning, Acton, MA) and grown for three days to a density of 80%. Three plasmids-(i) an AAV Helper plasmid containing the Rep and Cap genes, (ii) an Adenovirus Helper plasmid containing the adenovirus helper genes, and (iii) a transgene plasmid containing the sequence to be packaged flanked by AAV2 inverted terminal repeats were transfected into the cells using calcium phosphate. After three days, the cells were harvested. The cells were then lysed by three rounds of freeze/thaw and the cell debris was removed by centrifugation. The rAAV was precipitated using polyethylene glycol. After resuspension, the virus was purified by ultracentrifugation overnight on a cesium chloride gradient. The virus was formulated by dialysis and then filter-sterilized. After adjusting the titer (virus genomes/ml) of all AAV batches by dilution with PBS+0.001% Pluronic F-68, the AAVs were aliquoted to single use doses and stored at-80° C. until use. After thawing, no refreezing was done.

### Example 3: Human iPSC-Derived Neuron Culture and ZFP-TF AAV Transduction

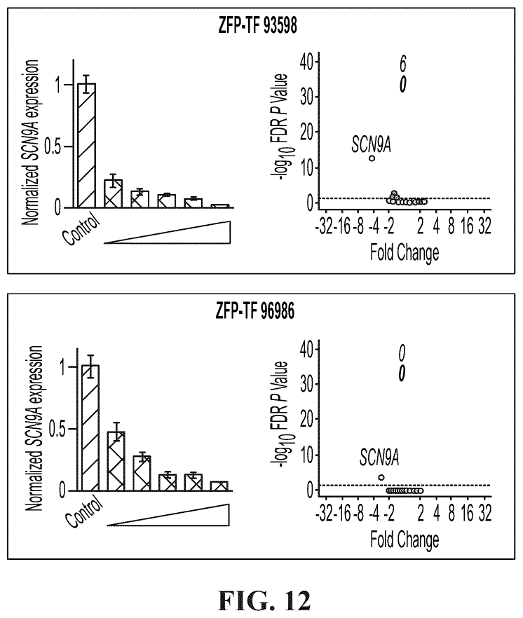

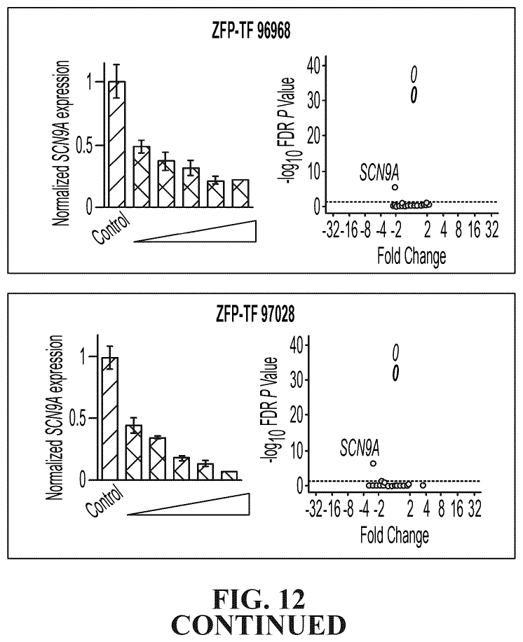

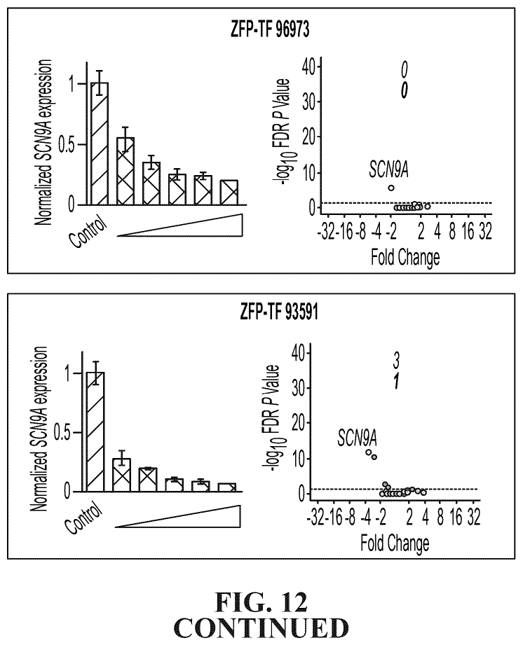

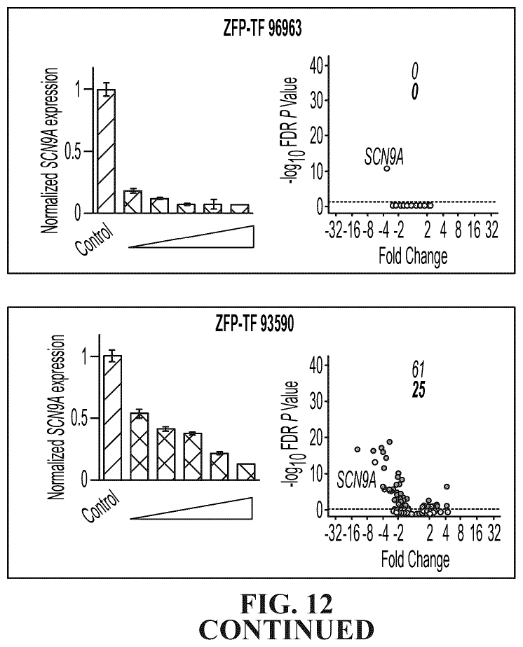

Human iPSC-derived GABAergic neurons were purchased from Cellular Dynamics International and plated onto poly-L-ornithine- and laminin-coated 96-well plates at a density of 40,000 cells per well and maintained according to the manufacturer's instructions. The cells were infected with AAV expressing the desired ZFP-TF at the indicated MOI 48 hours after plating and maintained for up to 32 days (50-75% media changes performed every 3-5 days). The cells were harvested at the end of the experimental period, RNA was isolated, and RT-qPCR was performed for gene expression analysis. FIG. 12 shows Nav1.7 expression levels in iPSC derived GABAergic neuronal culture following ZFP-TF AAV injection. For microarray analysis, the cells were transfected with 1E5 VGs/cell 48 hours after plating and harvested 32 days after viral transfection.

### Example 4: Human iPSC-Derived Sensory Neuron Progenitor Culture and ZFP-TF AAV Infection

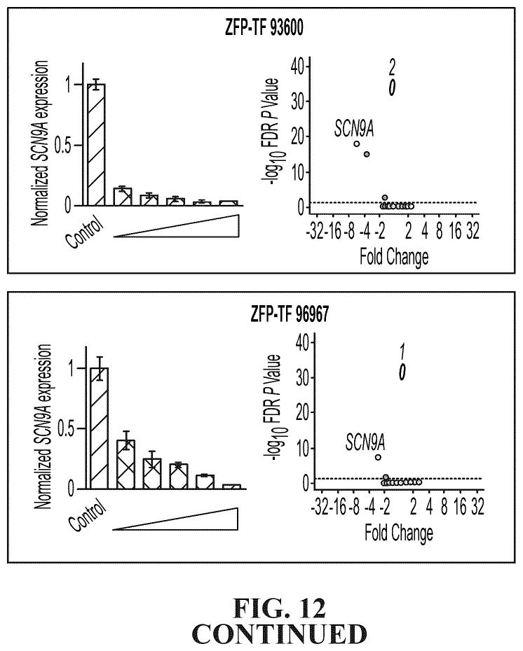

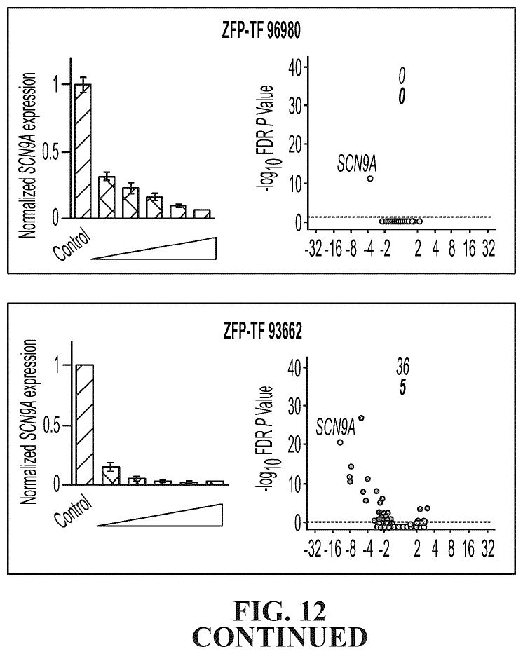

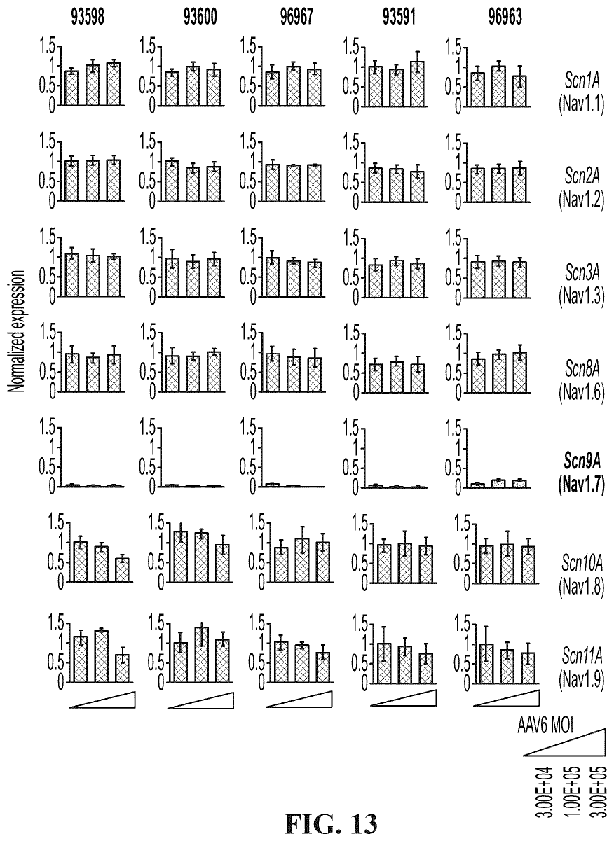

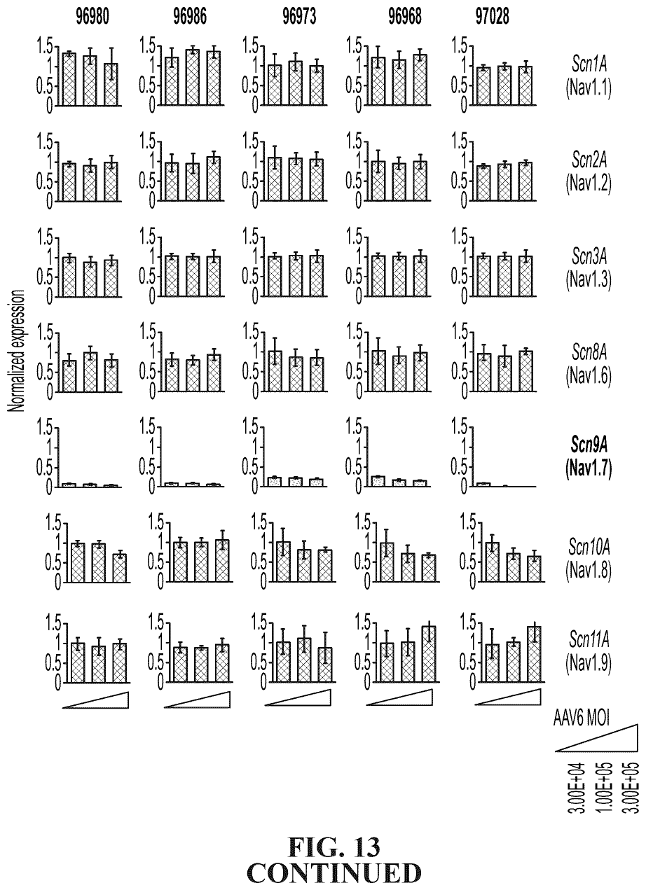

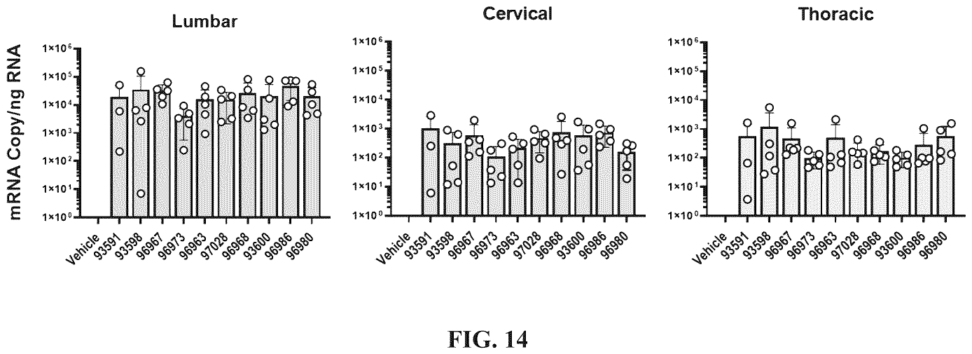

Human iPSC-derived sensory neuron progenitor cells were purchased from Axol Biosciences. Cells were cultured and differentiated on poly-D-lysine (PDL)-coated plates according to the manufacturer protocol. Cells were differentiated for 10 days and then infected with AAV expressing the desired ZFP-TF at the indicated MOIs. Cells were harvested on day 18 and RNA was isolated, and RT-qPCR was performed for gene expression analysis. FIG. 13 shows the expression levels of SCN9A and other Nav channels (SCN1A, SCN2A, SCN3A, SCN8A, SCN10A, and SCN11A) in progenitor sensory neurons following infection by AAV6-ZFP-TF.

### Example 5: Primary Mouse Neuron Culture and ZFP-TF AAV Infection

Primary mouse cortical neurons (MCNs) were purchased from Gibco. Cells were plated onto poly-D-lysine (PDL)-coated 96- or 24-well plates at 50,000 or 200,000 cells/well, respectively, and maintained according to the manufacturer's specifications using Gibco Neurobasal Medium containing GlutaMAX™ I supplement, B27 supplement, and penicillin/streptomycin. 48 hours after plating (at DIV2), 50,000 cells/well in 96-well plates were infected with AAV-ZFP at the indicated MOIs and harvested 7 days later (at DIV9; 50% media exchanges performed every 3-4 days) followed by RNA isolation and gene expression analysis by RT-qPCR. Alternatively, 200,000 neurons/well in 24-well plates were treated with 1E5 VGs/cell to ensure a 100% transduction rate and processed in a similar fashion for microarray analysis at DIV9.

### Example 6: Off-Target Activity of Anti-SCN9A ZFP-TFs

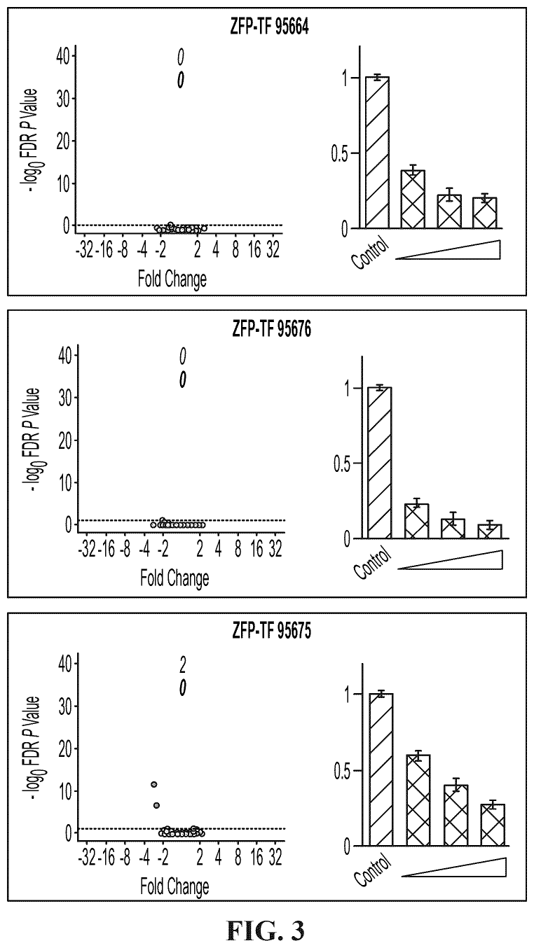

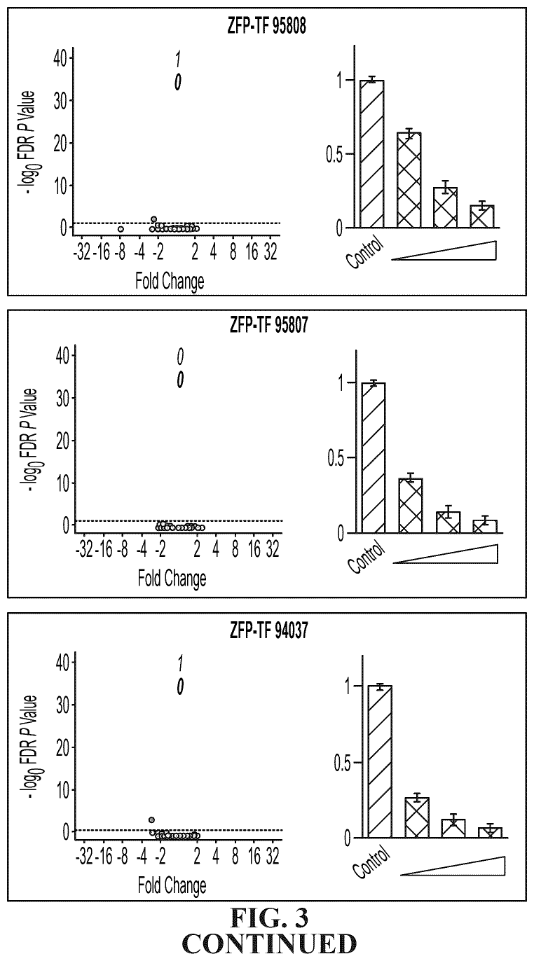

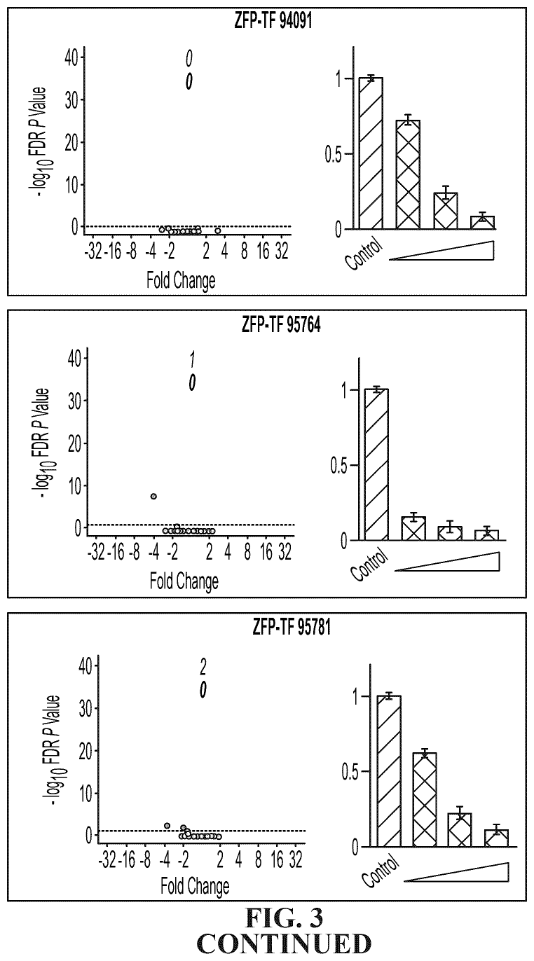

To evaluate the off-target impact of the ZFP-TFs on global gene expression, we performed microarray (Clariom S Array) experiments on total RNA isolated from human iPSC-derived neurons and primary mouse cortical neurons treated with AAVs encoding representative ZFP-TFs. Microarray analyses were performed following the manufacturer's protocol (Thermo Fisher Scientific), and the assay results were analyzed using TAC4 software. Apparent off-targets with FDR-corrected p-values≤0.05 were further investigated using RT-qPCR analysis. FIG. 3 shows the microarray results of ZFP-TFs targeting mice Scn9a gene in primary mouse cortical neurons. FIG. 12 shows microarray results of ZFP-TFs targeting human SCN9A gene in human iPSC-derived GABAergic neurons transduced with ZFP-TFs.

### Example 7: In Vivo Proof of Concept Study in Wildtype Mice

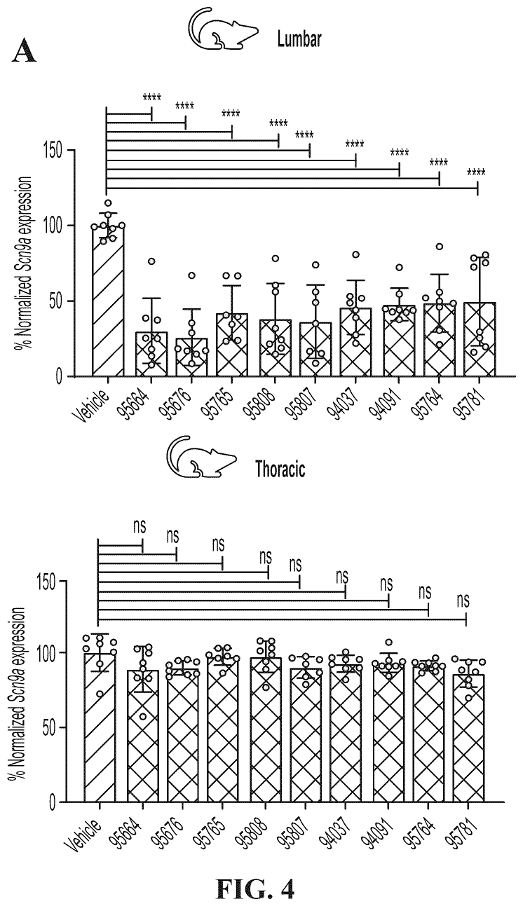

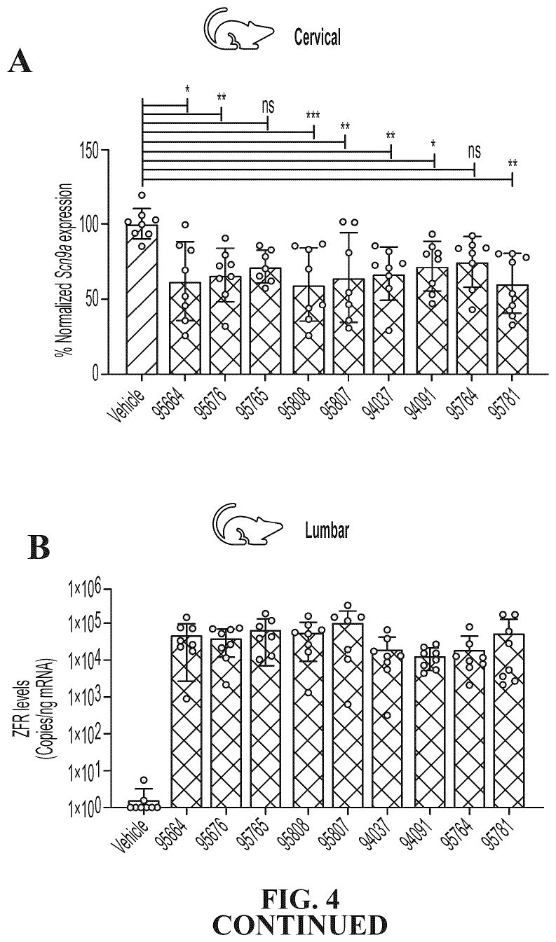

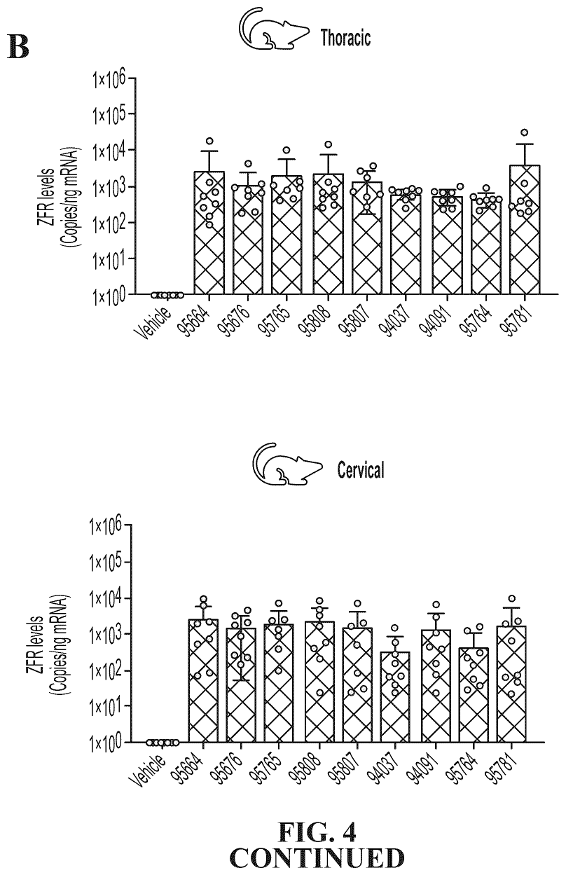

The pharmacological activities of the AAV9 delivered ZFP-TFs targeting mouse Scn9a gene in mice were evaluated in C57BL/6 (WT) mouse DRG neurons following intrathecal-lumbar (IT-L) injection. Transgene expression (ZFP-TF) and the expression level of the Scn9a gene were assessed in lumbar, cervical, and thoracic DRGs (FIG. 4).

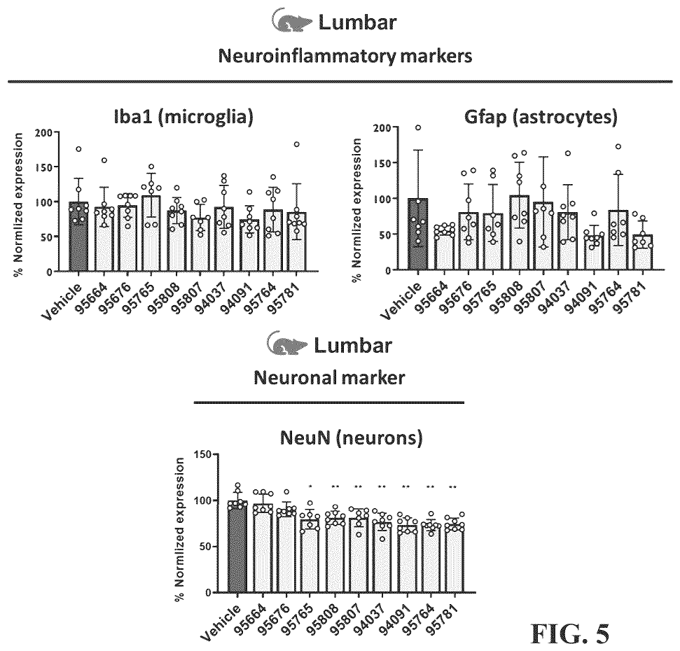

Neuroinflammatory (Gfap, Iba1) and neuronal loss (NeuN) markers expression were also evaluated in the lumbar DRG (which had the highest transgene expression and the most Scn9a repression) (FIG. 5). To harvest the tissue for subsequent analysis, mice were perfused with PBS, and the DRGs were extracted and dissected on ice. The tissue was then flash-frozen in liquid nitrogen and maintained at −80° C. until analysis. Reverse transcription was performed using the High-Capacity RT Kit (Thermo Fisher Scientific) kit following the manufacturer's instructions. TaqMan quantitative polymerase chain reaction (qPCR) was used to measure the expression levels of the ZFP-TFs, Scn9a, Gfap, Iba1, and NeuN. Gene expression levels were normalized to the geometric mean of the expression levels of the housekeeping genes Atp5b and Eif4a2. Vehicle treatment (VEH) was used as negative control. Overall, no neuronal loss or increase in neuroinflammation markers were observed.

The results are consistent with the in vitro Scn9a repression data obtained from mouse neuron culture (N2A cells). Scn9a mRNA levels were significantly reduced in lumbar DRG following treatment with any of the ZFP-TFs. There was minimal repression of Scn9a mRNA (30% repression compared to vehicle group) in cervical DRG in response to all test articles, while the expression levels of Scn9a mRNA did not change (compared to vehicle group) in thoracic DRG in response to any of the test articles. This was expected based on the absolute measurements of ZFP-TF mRNA levels in each DRG level. Lumbar DRG contained ~100-fold more ZFP-TF compared to cervical and thoracic DRGs.

Neuroinflammatory and neuronal marker transcript levels were also assessed as secondary endpoints. No significant upregulation of the neuroinflammatory markers (Iba1 and Gfap), but a minimum downregulation of neuronal markers (NeuN) was observed following intrathecal injection of the ZFP-TFs at the 2E+11 vg/mouse dose.

### Example 8: Spared Nerve Injury (SNI) Neuropathic Pain Model

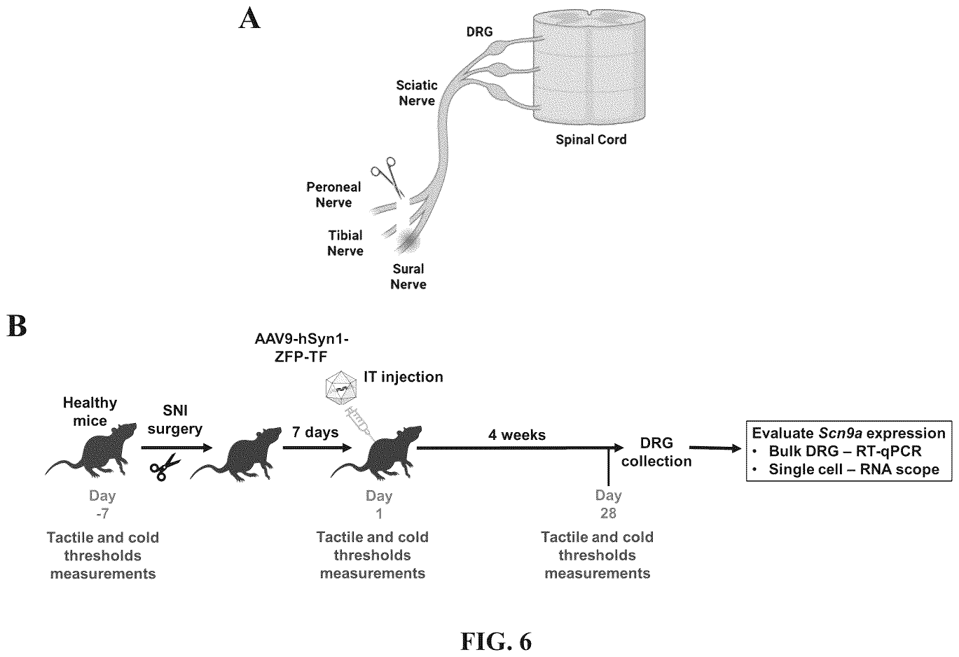

Spared Nerve Injury (SNI) is the most validated mouse neuropathic pain model and is considered to be the Gold standard to evaluate neuropathic pain in rodents. The model is generated surgically by cutting the tibial and peroneal nerves, and sparing the sural nerve (FIG. 6A). The damage to tibial and peroneal nerves results in marked hypersensitivity in the lateral area of the paw, which is innervated by the spared sural nerve. The advantages of the SNI model are the robustness of the response and that it doesn't require expert microsurgical skills.

### Example 9: In Vivo Evaluation of Pain Behavior Efficacy in Neuropathic Pain Mouse Model

The overall goal of the study was to evaluate the pharmacological activity and behavioral efficacy of AAV9 delivered ZFP-TFs targeting Scn9a via IT-L administration in male and female C57BL/6 (WT) mouse DRGs using the SNI model. The objectives of this study were to evaluate pharmacology and efficacy of four representative ZFP-TFs in repressing the Scn9a gene and reducing pain in a mouse neuropathic pain model. The main points to be evaluated were von Frey filaments (mechanical allodynia) and cold (acetone) stimuli (cold allodynia). All animals underwent the SNI surgery on day 7. Seven days post-SNI surgery, animals were evaluated for their responsiveness to mechanical (von Frey filaments) and cold stimuli and responsive animals were assigned to their respective groups as explained in the schematic diagram of FIG. 6B. This day was designated as day 1 of the study and the animals were administered a single bolus injection (10 μL) on the same day. Intrathecal (IT) administration did not involve surgical procedures and was directly administered by injection to lumbar (L) regions L3/L4 or L4/L5. The group assignments were as follows: Group 1 (Sham, no treatment), Group 2 (Vehicle Control), Group 3 (Gabapentin [GBP], 50 mg/kg, common anti-neuropathic drug), Group 4 (ZFP-TF 94091; 6.81E+11vg/mouse), Group 5 (ZFP-TF 95664; 8.12E+11 vg/mouse), Group 6 (ZFP-TF 95676; 7.99E+11 vg/mouse) and Group 7 (ZFP-TF 95807; 3.36E+11 vg/mouse). The sham operated group underwent surgery, but nerves were not cut (only skin opened and closed) and they did not receive any test article or vehicle. GBP was administered intraperitoneally (IP) approximately one hour prior to testing on each assessment. The doses for ZFP-TFs were based on maximum achievable concentration, based on the manufacturing capabilities and dose volume constraints.

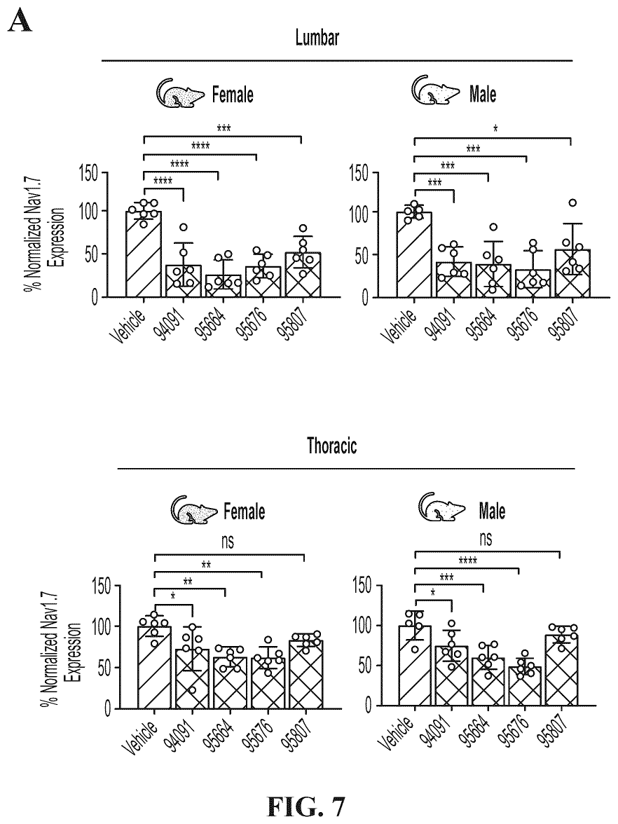

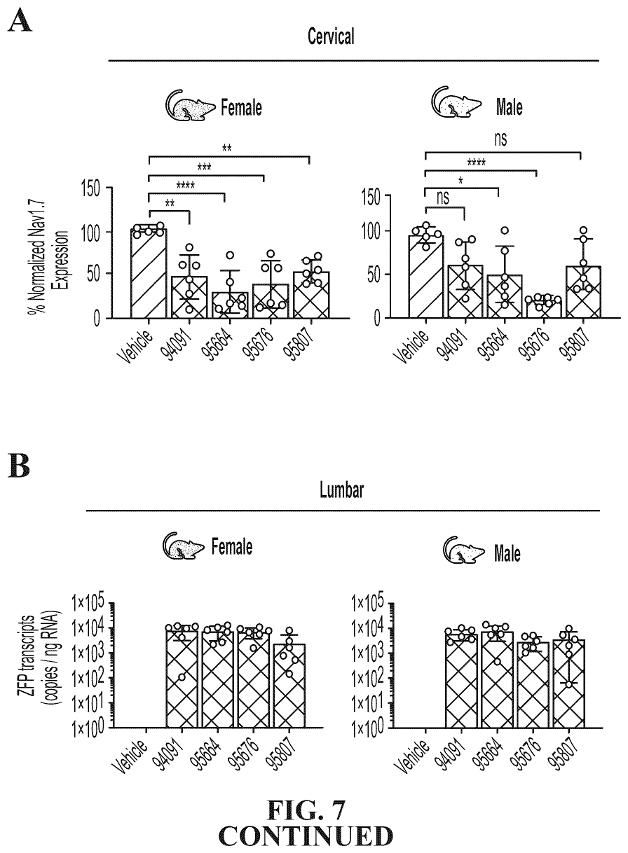

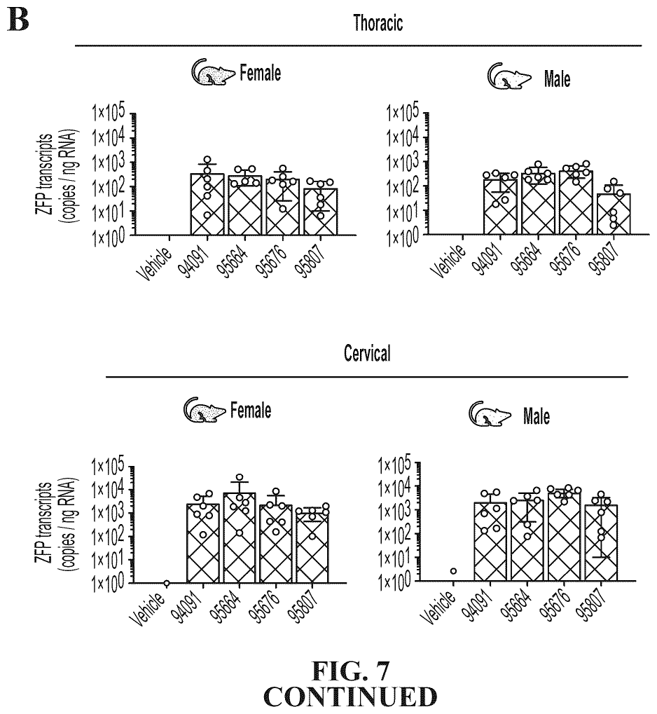

Scn9a mRNA expression levels were assessed in DRGs collected from lumbar, cervical, and thoracic levels on day 29 of the experiment from both male and female mice (FIG. 7A). Data were collected from combining three pairs of DRGs from each level following treatment with AAV9-ZFP-TF. ZFP-TFs were encoded under the human synapsin 1 promoter. Vehicle treatment was used as negative control. A significant reduction of Scn9a was observed in different DRG levels for both male and female animals.

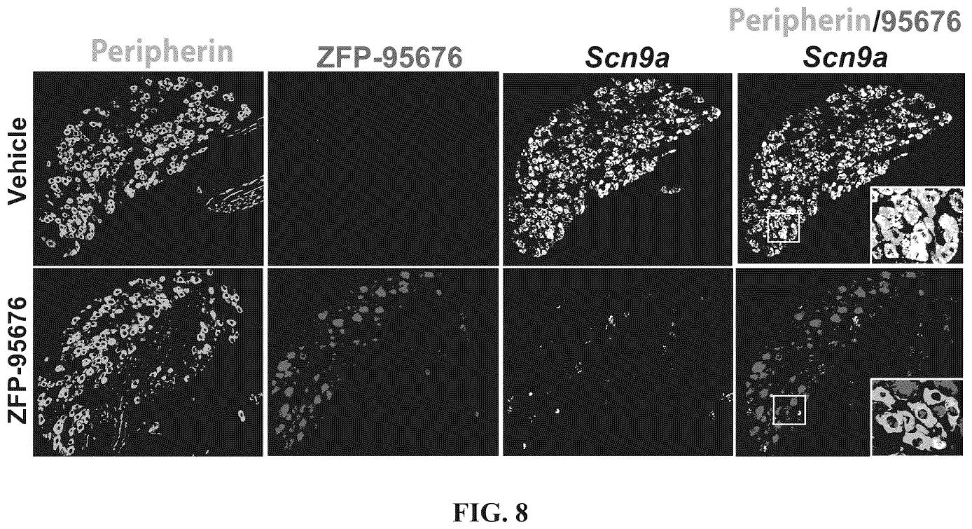

Some DRGs from the lumbar region of the animals treated with the ZFP-TF 95676 were paraffin embedded and processed for single cell analysis using to evaluate the ZFP-TF expression and Scn9a repression on single cell levels within the nociceptors. RNAscope in situ hybridization in combination with immunohistochemistry was used to assess the Scn9a mRNA levels and identify the nociceptors with a peripherin specific antibody, respectively. A high level of Scn9a was observed in peripherin positive cells in lumbar DRG isolated from the vehicle group. A significant reduction of Scn9a mRNA transcript was observed in peripherin positive/ZFP-TF positive cells in 95676 treated animals, illustrating that the AAV9-95676 significantly reduces the expression of Sen9a transcript in nociceptors (FIG. 8).

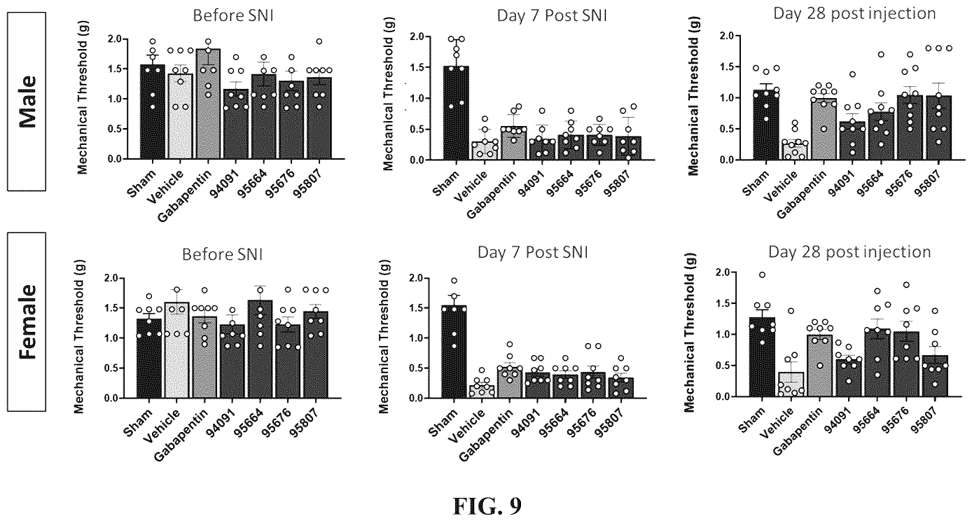

Animal response to mechanical allodynia 28 days after injection of representative ZFP-TFs post SNI surgery was assessed (FIG. 9). By day 28 post-dose, the increase in mechanical threshold (MT) was at the level of GBP for both ZFP-TF 95664 and ZFP-TF 95676, and was statistically significant compared with the control group. No significant difference was noted in MT compared with the control group for females treated with ZFP-TF 94091. Although administered at approximately half the dose compared to the other ZFP-TFs, 95807 resulted in higher MT values than the control. The MT responses in males were similar to females for ZFP-TF 94091, 95664, and 95676. However, unlike females, males treated with ZFP-TF 95807, administered at half the dose, demonstrated increase in MT on day 28 similar to GBP and these differences were statistically significant compared with the control group.

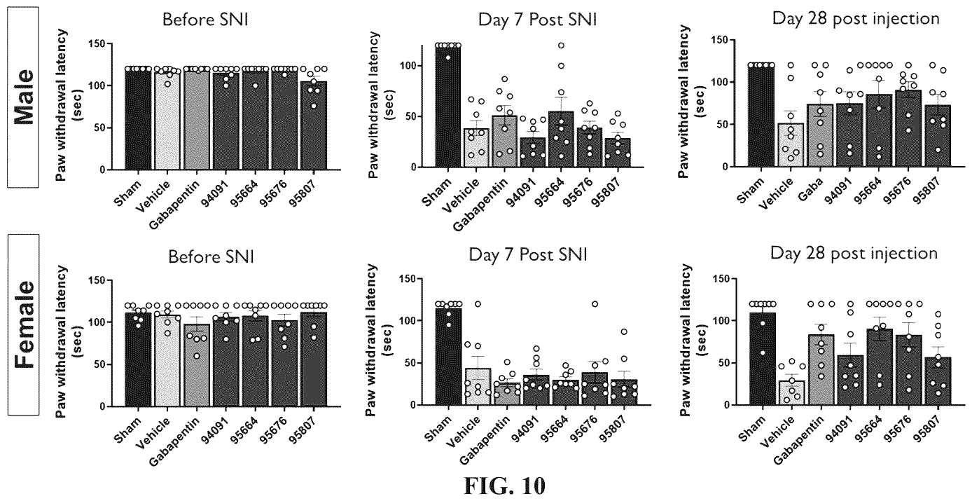

Animal response to cold allodynia 28 days after injection of representative ZFP-TFs post SNI surgery was assessed (FIG. 10). Female mice treated with ZFP-TFs also responded to cold allodynia as measured by the PWL (paw withdrawal latency) in seconds and similar to mechanical allodynia, females treated with ZFP-TFs 95664 and 95676 had the highest PWL responses by day 28, which were statistically significant compared with the control group and at the level of GBP. Male response to cold allodynia appeared to be highly variable for all groups including GBP and control groups; however, the trend in response appeared to be similar to mechanical allodynia with highest PWL responses noted for males treated with ZFP-TF 95664 followed by ZFP-TFs 95676 and 95807.

### Example 10: In Vivo Evaluation of Possible Intrinsic Neurotoxicity in Wildtype Mice

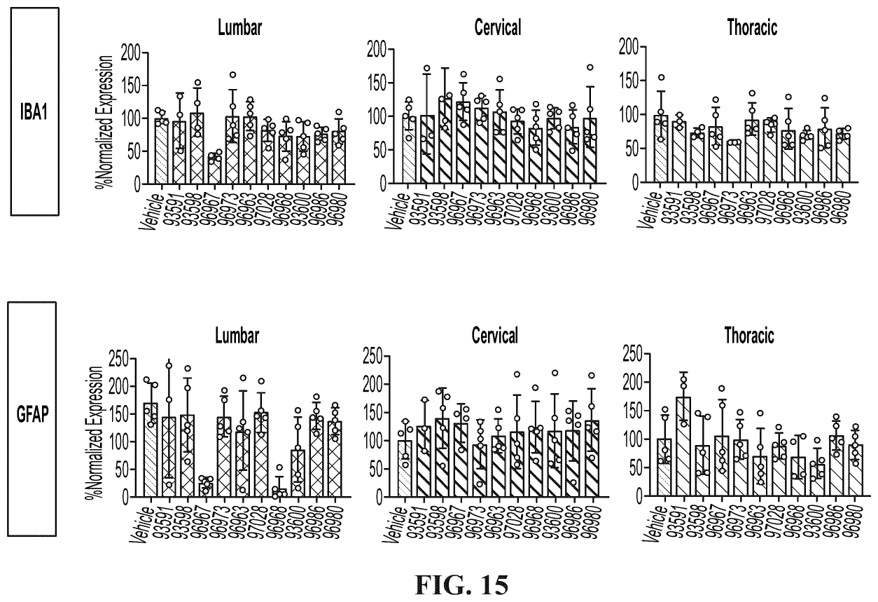

The intrinsic toxicity of the AAV9 delivered ZFP-TFs targeting human SCN9A gene in mice were evaluated in C57BL/6 (WT) mouse DRG neurons following intrathecal (IT) injection. Transgene expression (ZFP-TF) expression was assessed in lumbar, cervical, and thoracic DRGs. Expression of neuroinflammatory (Gfap, Iba1) and neuronal loss (NeuN) markers was also evaluated in the lumbar, cervical, and thoracic DRGs. FIG. 15. shows that no significant upregulation of the neuroinflammatory markers (Iba1 and Gfap), or downregulation of neuronal markers (NeuN) was observed following intrathecal injection of the ZFP-TFs at the 2E+11 vg/mouse dose.

### Example 11: In Vivo Evaluation of Target Engagement and Safety of ZFP-TF Targeting Human SCN9A in Nonhuman-Primates (NHP)

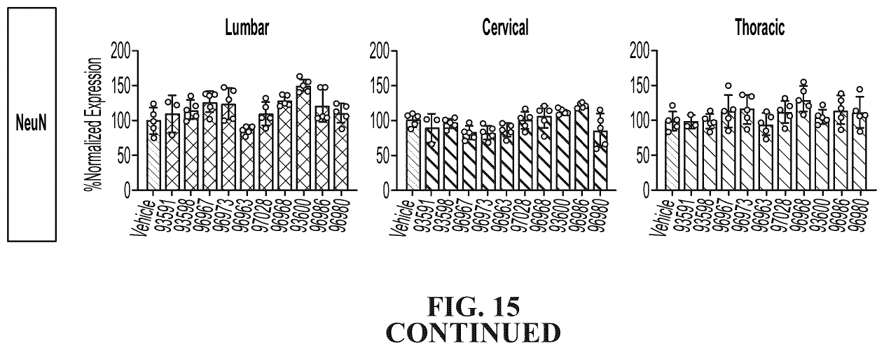

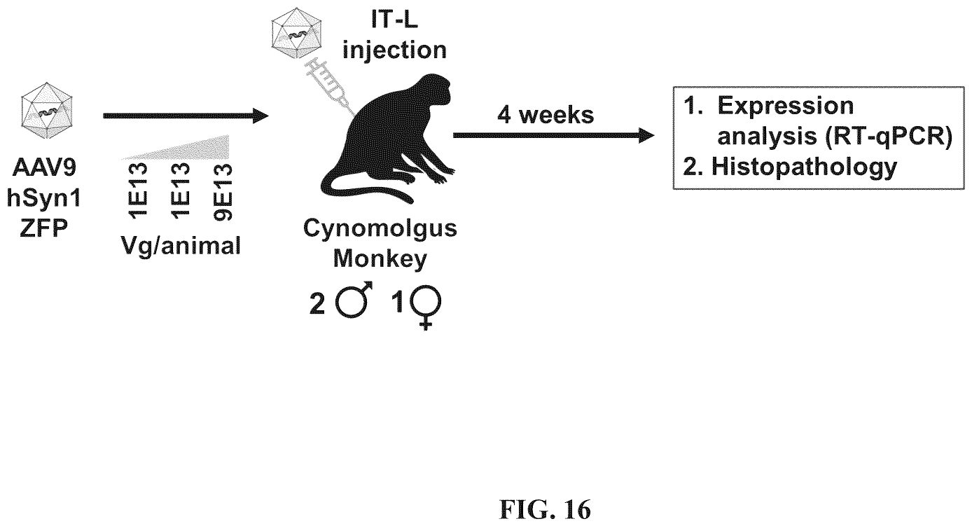

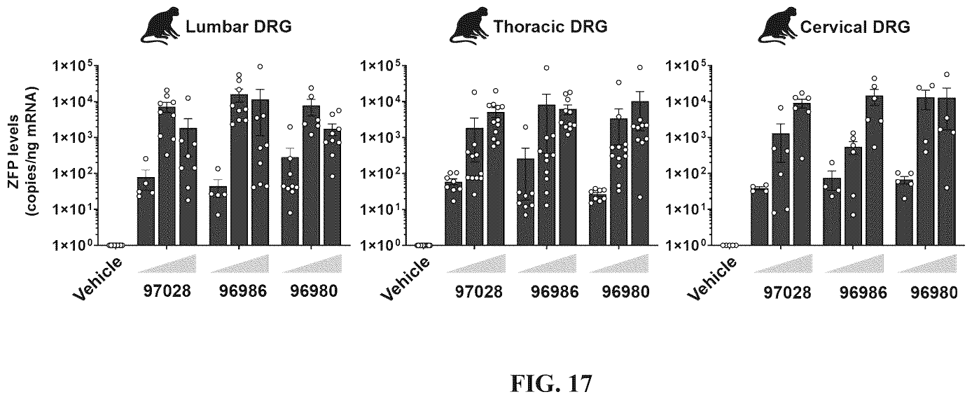

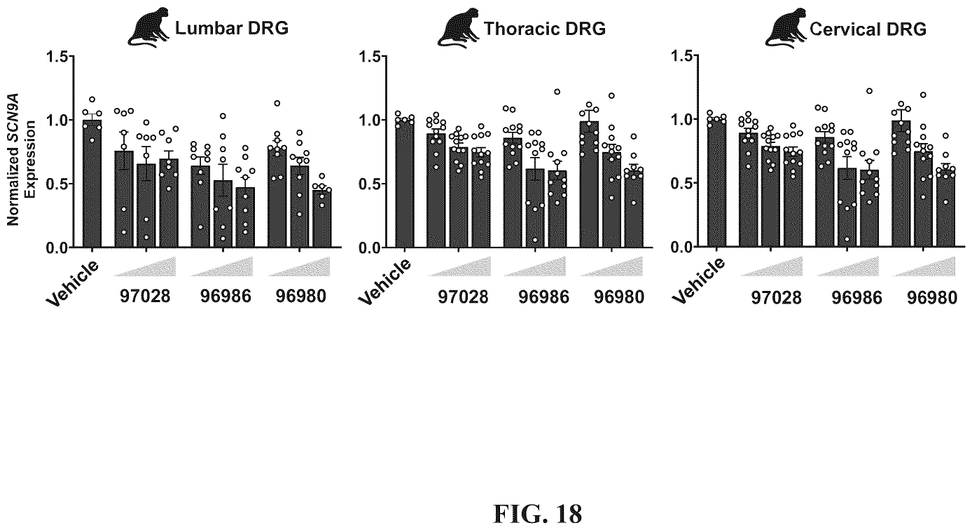

ZFP-TFs can bind to the homologues sequence within the NHP SCN9A TSS, so the pharmacology and target specificity of the AAV9-delivered ZFP-TFs were evaluated in cynomolgus monkeys in a 4-weeks dose range-finding (DRF) pharmacology and toxicology study following a single IT-L administration at three dose levels, 1E12 (low-dose), 1E13 (mid-dose), and 9E13 (high-dose) vg/animal (FIG. 16). DRGs were collected from lumbar (L5 and L1), thoracic (T7, T6, and T1), and cervical (C6 and C5) levels. The expression level of ZFP-TFs and SCN9A were evaluated using RT-qPCR. AAV9 delivered ZFP-TFs were expressed in a dose-dependent manner in all DRG regions analyzed (FIG. 17). ZFP-TFs 96986 and 96980 repressed SCN9A transcript at each region by 40-60% at the bulk tissue level across a 100-fold dose range compared to the vehicle group while ZFP-TF 97028 exhibited less repression levels (FIG. 18). Altogether, the data collected from the NHPs confirmed pharmacology and specificity of AAV9-ZFR-F in this preclinical species.

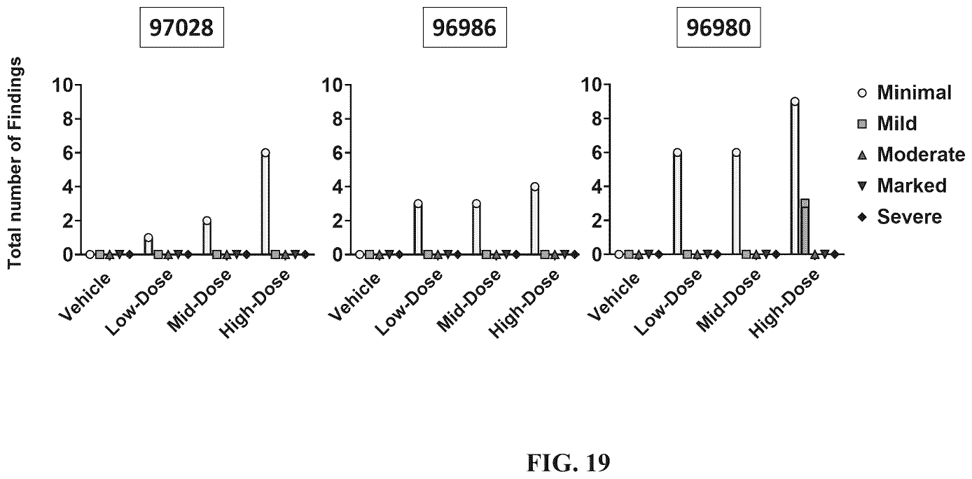

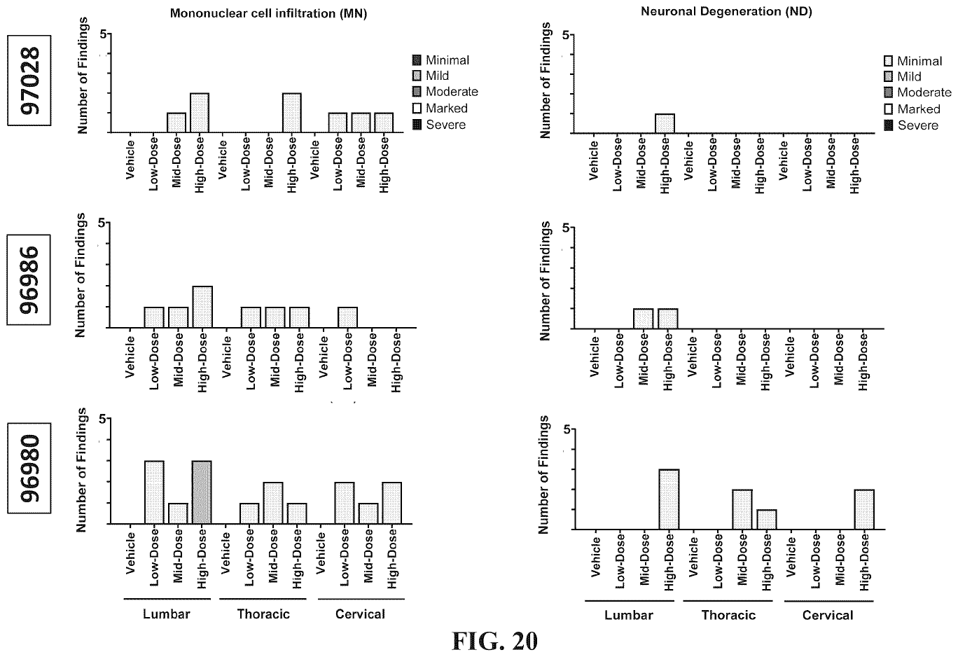

All NHPs survived until their scheduled necropsy. There were no abnormal clinical signs or behaviors, body weight, clinical pathology (hematology, chemistry, and coagulation), and necropsy findings that could be attributed to the treatment with ZFP-TFs. Liver panel was included in the clinical pathology evaluations and showed no abnormalities. Histopathology was performed for the following tissues: adrenal glands, brain, DRGs, epididymites, heart, kidney, small and large intestines, liver, lung, ovary, pancreases, sciatic nerve, skeletal muscle, spinal cord (lumbar, thoracic, cervical), spleen, stomach, testes, thymus, and trigeminal ganglia. Among all three NHPs, abnormal findings with minimal to mild severity were observed only for DRGs at all levels. The findings included mononuclear cell infiltration (MN) and neuronal degeneration (ND), with most of the findings being minimal and only 3 mild incidents in lumber DRG in the high dose group (FIG. 19). Most of the findings were in NHPs treated with ZFP-TF 96980. AAV9-96980 related minimal MN was observed in cervical DRG in low-(67%), mid-(33%), and high-dose (67%) groups. In thoracic DRG minimal MN was observed in low-(33%), mid-(67%), and high-dose (33%) groups, and in lumbar DRG in low-(100%), and mid-dose (33%) groups. 96980-related mild MN was only observed in the lumbar DRG of all three NHPs in high-dose group (FIG. 20). For NHPs treated with AAV9-96980, minimal MN was observed in lumbar DRG in low (33%), mid (33%) and high-dose (66%) groups in lumbar and thoracic DRGs. In general, almost all findings were of minimal severity except for the finding of mild MN in lumbar DRG of the high-dose group (FIG. 20). Despite microscopic findings in various peripheral tissues, no abnormal behavior indicative of neurological dysfunction was exhibited in any animal.

What is claimed is:

1. A fusion protein comprising a zinc finger protein (ZFP) domain and a transcription repressor domain, wherein the ZFP domain binds to a target sequence of a human SCN9A gene, wherein the target sequence comprises any one of SEQ ID NOs: 16-24.

2. (canceled)

3. (canceled)

4. The fusion protein of claim 1, wherein the fusion protein represses expression of the SCN9A gene by at least about 40%, 75%, 90%, 95%, or 99% with no or minimal detectable off-target binding or activity.

5. The fusion protein of claim 1, wherein the transcription repressor domain comprises a KRAB domain, wherein the KRAB domain optionally is from a human KOX1 protein.

6. The fusion protein of claim 1, wherein the ZFP domain is linked to the transcription repressor domain through a peptide linker.

7. The fusion protein of claim 1, wherein the ZFP domain comprises a DNA-binding recognition helix sequence shown in the table below wherein the sequences identification numbers are shown in parentheses:
DNA-Recognition Helix Amino Acid
Sequence Within Each Finger

| F1 | F2 | F3 | F4 | F5 | F6 |
|---|---|---|---|---|---|
| DRSDLSR | QSGSLTR | QSGDLTR | DSSNRAK | DRSHLSR | TSGHLSR |
| (25) | (26) | (27) | (28) | (29) | (30) |
| ASKTRTN | QSGSLTR | QSGDLTR | DSSNRAK | DRSHLTR | TSGHLSR |
| (31) | (26) | (27) | (28) | (32) | (30) |
| ASKTRTN | QSGSLTR | QSGDLTR | DSSNRAK | DSSHRTR | |
| (31) | (26) | (27) | (28) | (33) | |
| ASKTRTN | QSGSLTR | QSGDLTR | DRSNLTR | DRSHLAR | |
| (31) | (26) | (27) | (34) | (35) | |
| ASKTRTN | QSGSLTR | QSGDLTR | DRSNLTR | DRSHLSR | TSGHLSR |
| (31) | (26) | (27) | (34) | (29) | (30) |
| DRSYRNT | RRSDLKR | ERGTLAR | DRSALAR | DRSHLTR | QSGDLTR |
| (36) | (37) | (38) | (39) | (32) | (27) |
| DRSNLSR | LKFALAN | RSDNLST | RSAALAR | RSDHLST | QSAHRIT |
| (40) | (41) | (42) | (43) | (44) | (45) |
| RSDDLSK | RSDHRTN | DRSHLTR | TSANLSR | RSDSLSR | DRSVRTK |
| (46) | (47) | (32) | (48) | (49) | (50) |
| YKHVLSD | TSGSLTR | RSDSLLR | NYASRTW | RSDSLLR | NYASRTW |
| (51) | (52) | (53) | (54) | (53) | (54) |
| RSDSLSQ | RKADRTR | QSGDLTR | LKDTLRR | RSANLAR | QSSDLRR |
| (55) | (56) | (27) | (57) | (58) | (59) |
| HKTSLKD | | QSNHLTE | QNATRTK | DRSALSR | RSDHLSR |
| (60) | | (61) | (62) | (63) | (64) |
| QSGDLTR | LKDTLRR | RSANLAR | QSSDLRR | LRHHLTR | LRHNLRA |
| (27) | (57) | (58) | (59) | (65) | (66) |
| \hat{ }ASKTRTN | QSGSLTR | \hat{ }QSGDLTR | DRSNLTR | DRSHLAR | |
| (67) | (26) | (68) | (34) | (35) | |
| \hat{ }DRSDLSR | QSGSLTR | QSGDLTR | DSSNRAK | DRSHLSR | TSGHLSR |
| (69) | (26) | (27) | (28) | (29) | (30) |
| \hat{ }DRSYRNT | RRSDLKR | \hat{ }ERGTLAR | DRSALAR | DRSHLTR | QSGDLTR |
| (70) | (37) | (71) | (39) | (32) | (27) |
| \hat{ }ASKTRTN | QSGSLTR | QSGDLTR | DSSNRAK | DSSHRTR | |
| (67) | (26) | (27) | (28) | (33) | |
| ASKTRTN | QSGSLTR | \hat{ }QSGDLTR | DSSNRAK | DSSHRTR | |
| (31) | (26) | (68) | (28) | (33) | |
| \hat{ }ASKTRTN | QSGSLTR | \hat{ }QSGDLTR | DRSNLTR | DRSHLSR | TSGHLSR |
| (67) | (26) | (68) | (34) | (29) | (30) |
| \hat{ }ASKTRTN | QSGSLTR | \hat{ }QSGDLTR | DSSNRAK | DRSHLTR | TSGHLSR |
| (67) | (26) | (68) | (28) | (32) | (30) |

8. The fusion protein of claim 7, wherein the ZFP domain comprises the DNA-binding recognition helix sequences as shown in a single row of the table in claim 7.

9. The fusion protein of claim 7, wherein the ZFP domain of the fusion protein
comprises five or six zinc fingers;
comprises the DNA-binding recognition helix sequences as shown in a single row of the table in claim 7 and are ordered from F1 to F6; and/or;
comprises the DNA-binding recognition helix sequences linked as shown in the table below wherein the DNA-binding recognition helix sequences are in boldface, intramodule and intermodule linkers are underlined and R (−5) Q backbone mutations are indicated by boldface and underline and the sequences identification numbers are shown in parentheses:

| SEQ ID NO | Amino Acid sequence (helix, ZF linker, & interdomain linker) |
|---|---|
| (72) | MAPKKKRKVGVPAAMAERPFQCRICMRNFSDRSDLSRHIRTHTGEKPFACDICGRKFAQSG |
| | SLTRHTKIHTGSQKPFQCRICMRNFSQSGDLTRHIRTHTGEKPFACDICGRKFADSSNRAKH |
| | TKIHTGSQKPFQCRICMRNFSDRSHLSRHIRTHTGEKPFACDICGRKFATSGHLSRHTKIHLR |
| | QKDAARGSGGDAKSLTAWSRTLVTFKDVFVDFTREEWKLLDTAQQIVYRNVMLENYKNL |
| | VSLGYQLTKPDVILRLEKGEEPWLVEREIHQETHPDSETAFEIKSSVRS |
| (73) | MAPKKKRKVGVPAAMAERPFQCRICMRNFSASKTRTNHIRTHTGEKPFACDICGRKFAQS |
| | GSLTRHTKIHTGSQKPFQCRICMRNFSQSGDLTRHIRTHTGEKPFACDICGRKFADSSNRAK |
| | HTKIHTGSQKPFQCRICMRNFSDRSHLTRHIRTHTGEKPFACDICGRKFATSGHLSRHTKIH |
| | LROKDAARGSGGDAKSLTAWSRTLVTFKDVFVDFTREEWKLLDTAQQIVYRNVMLENYK |
| | NLVSLGYQLTKPDVILRLEKGEEPWLVEREIHQETHPDSETAFEIKSSVRS |
| (74) | MAPKKKRKVGVPAAMAERPFQCRICMRNFSASKTRTNHIRTHTGEKPFACDICGRKFAQS |
| | GSLTRHTKIHTGSQKPFQCRICMRNFSQSGDLTRHIRTHTGEKPFACDICGRKFADSSNRAK |
| | HTKIHTGEKPFQCRICMRKFADSSHRTRHTKIHLROKDAARGSGGDAKSLTAWSRTLVTFK |
| | DVFVDFTREEWKLLDTAQQIVYRNVMLENYKNLVSLGYQLTKPDVILRLEKGEEPWLVER |
| | EIHQETHPDSETAFEIKSSVRS |
| (76) | MAPKKKRKVGVPAAMAERPFQCRICMRNFSASKTRTNHIRTHTGEKPFACDICGRKFAQS |
| | GSLTRHTKIHTGSQKPFQCRICMRNFSQSGDLTRHIRTHTGEKPFACDICGRKFADRSNLTR |
| | HTKIHTGEKPFQCRICMRKFADRSHLARHTKIHLRQKDAARGSGGDAKSLTAWSRTLVTFK |
| | DVFVDFTREEWKLLDTAQQIVYRNVMLENYKNLVSLGYQLTKPDVILRLEKGEEPWLVER |
| | EIHQETHPDSETAFEIKSSVRS |
| (77) | MAPKKKRKVGVPAAMAERPFQCRICMRNFSASKTRTNHIRTHTGEKPFACDICGRKFAQS |
| | GSLTRHTKIHTGSQKPFQCRICMRNFSQSGDLTRHIRTHTGEKPFACDICGRKFADRSNLTR |
| | HTKIHTGSQKPFQCRICMRNFSDRSHLSRHIRTHTGEKPFACDICGRKFATSGHLSRHTKIH |
| | LROKDAARGSGGDAKSLTAWSRTLVTFKDVFVDFTREEWKLLDTAQQIVYRNVMLENYK |
| | NLVSLGYQLTKPDVILRLEKGEEPWLVEREIHQETHPDSETAFEIKSSVRS |
| (78) | MAPKKKRKVGVPAAMAERPFQCRICMRNFSDRSYRNTHIRTHTGEKPFACDICGRKFARR |
| | SDLKRHTKIHTHPRAPIPKPFQCRICMRNFSERGTLARHIRTHTGEKPFACDICGRKFADRS |
| | ALARHTKIHTHPRAPIPKPFQCRICMRNFSDRSHLTRHIRTHTGEKPFACDICGRKFAQSGD |
| | LTRHTKIHLROKDAARGSGGDAKSLTAWSRTLVTFKDVFVDFTREEWKLLDTAQQIVYRN |
| | VMLENYKNLVSLGYQLTKPDVILRLEKGEEPWLVEREIHQETHPDSETAFEIKSSVRS |
| (79) | MAPKKKRKVGVPAAMAERPFQCRICMRNFSDRSNLSRHIRTHTGEKPFACDICGRKFALKF |
| | ALANHTKIHTGSQKPFQCRICMRNFSRSDNLSTHIRTHTGEKPFACDICGRKFARSAALARH |
| | TKIHTGSQKPFQCRICMRNFSRSDHLSTHIRTHTGEKPFACDICGRKFAQSAHRITHTKIHLR |
| | QKDAARGSGGDAKSLTAWSRTLVTFKDVFVDFTREEWKLLDTAQQIVYRNVMLENYKNL |
| | VSLGYQLTKPDVILRLEKGEEPWLVEREIHQETHPDSETAFEIKSSVRS |
| (80) | MAPKKKRKVGVPAAMAERPFQCRICMRNFSRSDDLSKHIRTHTGEKPFACDICGRKFARSD |
| | HRTNHTKIHTGSQKPFQCRICMRNFSDRSHLTRHIRTHTGEKPFACDICGRKFATSANLSRH |
| | TKIHTGSQKPFQCRICMRNFSRSDSLSRHIRTHTGEKPFACDICGRKFADRSVRTKHTKIHLR |
| | QKDAARGSGGDAKSLTAWSRTLVTFKDVFVDFTREEWKLLDTAQQIVYRNVMLENYKNL |
| | VSLGYQLTKPDVILRLEKGEEPWLVEREIHQETHPDSETAFEIKSSVRS |
| (81) | MAPKKKRKVGVPAAMAERPFQCRICMRNFSYKHVLSDHIRTHTGEKPFACDICGRKFATS |
| | GSLTRHTKIHTHPRAPIPKPFQCRICMRNFSRSDSLLRHIRTHTGEKPFACDICGRKFANYAS |
| | RTWHTKIHTGSQKPFQCRICMRNFSRSDSLLRHIRTHTGEKPFACDICGRKFANYASRTWH |
| | TKIHLROKDAARGSGGDAKSLTAWSRTLVTFKDVFVDFTREEWKLLDTAQQIVYRNVMLE |
| | NYKNLVSLGYQLTKPDVILRLEKGEEPWLVEREIHQETHPDSETAFEIKSSVRS |
| (82) | MAPKKKRKVGVPAAMAERPFQCRICMRNFSRSDSLSQHIRTHTGEKPFACDICGRKFARK |
| | ADRTRHTKIHTGSQKPFQCRICMRNFSQSGDLTRHIRTHTGEKPFACDICGRKFALKDTLR |
| | RHTKIHTGSQKPFQCRICMRNFSRSANLARHIRTHTGEKPFACDICGRKFAQSSDLRRHTKI |
| | HLROKDAARGSGGDAKSLTAWSRTLVTFKDVFVDFTREEWKLLDTAQQIVYRNVMLENY |
| | KNLVSLGYQLTKPDVILRLEKGEEPWLVEREIHQETHPDSETAFEIKSSVRS |
| (83) | MAPKKKRKVGVPAAMAERPFQCRICMRKFAHKTSLKDHTKIHTGEKPFQCRICMRNFSQS |
| | NHLTEHIRTHTGEKPFACDICGRKFAQNATRTKHTKIHTGSQKPFQCRICMRNFSDRSALS |
| | RHIRTHTGEKPFACDICGRKFARSDHLSRHTKIHLRQKDAARGSGGDAKSLTAWSRTLVTF |
| | KDVFVDFTREEWKLLDTAQQIVYRNVMLENYKNLVSLGYQLTKPDVILRLEKGEEPWLVE |
| | REIHQETHPDSETAFEIKSSVRS |
| (84) | MAPKKKRKVGVPAAMAERPFQCRICMRNFSQSGDLTRHIRTHTGEKPFACDICGRKFALK |
| | DTLRRHTKIHTGSQKPFQCRICMRNFSRSANLARHIRTHTGEKPFACDICGRKFAQSSDLRR |
| | HTKIHTGSQKPFQCRICMRNFSLRHHLTRHIRTHTGEKPFACDICGRKFALRHNLRAHTKI |
| | HLRQKDAARGSGGDAKSLTAWSRTLVTFKDVFVDFTREEWKLLDTAQQIVYRNVMLENY |
| | KNLVSLGYQLTKPDVILRLEKGEEPWLVEREIHQETHPDSETAFEIKSSVRS |
| (85) | MAPKKKRKVGVPAAMAERPFQCRICMQNFSASKTRTNHIRTHTGEKPFACDICGRKFAQS |
| | GSLTRHTKIHTGSQKPFQCRICMQNFSQSGDLTRHIRTHTGEKPFACDICGRKFADRSNLT |
| | RHTKIHTGEKPFQCRICMRKFADRSHLARHTKIHLRQKDAARGSGGDAKSLTAWSRTLVT |
| | FKDVFVDFTREEWKLLDTAQQIVYRNVMLENYKNLVSLGYQLTKPDVILRLEKGEEPWLV |
| | EREIHQETHPDSETAFEIKSSVRS |
| (86) | MAPKKKRKVGVPAAMAERPFQCRICMQNFSDRSDLSRHIRTHTGEKPFACDICGRKFAQS |
| | GSLTRHTKIHTGSQKPFQCRICMRNFSQSGDLTRHIRTHTGEKPFACDICGRKFADSSNRAK |
| | HTKIHTGSQKPFQCRICMRNFSDRSHLSRHIRTHTGEKPFACDICGRKFATSGHLSRHTKIH |
| | LROKDAARGSGGDAKSLTAWSRTLVTFKDVFVDFTREEWKLLDTAQQIVYRNVMLENYK |
| | NLVSLGYQLTKPDVILRLEKGEEPWLVEREIHQETHPDSETAFEIKSSVRS |
| (87 | MAPKKKRKVGVPAAMAERPFQCRICMQNFSDRSYRNTHIRTHTGEKPFACDICGRKFARR |
| | SDLKRHTKIHTHPRAPIPKPFQCRICMQNFSERGTLARHIRTHTGEKPFACDICGRKFADRS |
| | ALARHTKIHTHPRAPIPKPFQCRICMRNFSDRSHLTRHIRTHTGEKPFACDICGRKFAQSGD |
| | LTRHTKIHLROKDAARGSGGDAKSLTAWSRTLVTFKDVFVDFTREEWKLLDTAQQIVYRN |
| | VMLENYKNLVSLGYQLTKPDVILRLEKGEEPWLVEREIHQETHPDSETAFEIKSSVRS |
| (88) | MAPKKKRKVGVPAAMAERPFQCRICMQNFSASKTRTNHIRTHTGEKPFACDICGRKFAQS |
| | GSLTRHTKIHTGSQKPFQCRICMRNFSQSGDLTRHIRTHTGEKPFACDICGRKFADSSNRAK |
| | HTKIHTGEKPFQCRICMRKFADSSHRTRHTKIHLRQKDAARGSGGDAKSLTAWSRTLVTFK |
| | DVFVDFTREEWKLLDTAQQIVYRNVMLENYKNLVSLGYQLTKPDVILRLEKGEEPWLVER |
| | EIHQETHPDSETAFEIKSSVRS |
| (89) | MAPKKKRKVGVPAAMAERPFQCRICMRNFSASKTRTNHIRTHTGEKPFACDICGRKFAQS |
| | GSLTRHTKIHTGSQKPFQCRICMQNFSQSGDLTRHIRTHTGEKPFACDICGRKFADSSNRA |
| | KHTKIHTGEKPFQCRICMRKFADSSHRTRHTKIHLROKDAARGSGGDAKSLTAWSRTLVTF |
| | KDVFVDFTREEWKLLDTAQQIVYRNVMLENYKNLVSLGYQLTKPDVILRLEKGEEPWLVE |
| | REIHQETHPDSETAFEIKSSVRS |
| (90) | MAPKKKRKVGVPAAMAERPFQCRICMQNFSASKTRTNHIRTHTGEKPFACDICGRKFAQS |
| | GSLTRHTKIHTGSQKPFQCRICMQNFSQSGDLTRHIRTHTGEKPFACDICGRKFADRSNLT |
| | RHTKIHTGSQKPFQCRICMRNFSDRSHLSRHIRTHTGEKPFACDICGRKFATSGHLSRHTKI |
| | HLRQKDAARGSGGDAKSLTAWSRTLVTFKDVFVDFTREEWKLLDTAQQIVYRNVMLENY |
| | KNLVSLGYQLTKPDVILRLEKGEEPWLVEREIHQETHPDSETAFEIKSSVRS |
| (91) | MAPKKKRKVGVPAAMAERPFQCRICMQNFSASKTRTNHIRTHTGEKPFACDICGRKFAQS |
| | GSLTRHTKIHTGSQKPFQCRICMQNFSQSGDLTRHIRTHTGEKPFACDICGRKFADSSNRA |
| | KHTKIHTGSQKPFQCRICMRNFSDRSHLTRHIRTHTGEKPFACDICGRKFATSGHLSRHTKI |
| | HLROKDAARGSGGDAKSLTAWSRTLVTFKDVFVDFTREEWKLLDTAQQIVYRNVMLENY |
| | KNLVSLGYQLTKPDVILRLEKGEEPWLVEREIHQETHPDSETAFEIKSSVRS |

10. A nucleic acid construct comprising a coding sequence for the fusion protein of claim 1, wherein the coding sequence is linked operably to a transcription regulatory element.

11. The nucleic acid construct of claim 10, wherein the transcription regulatory element is a mammalian promoter that is constitutively active or inducible in neurons, wherein the construct is optionally a recombinant viral construct.

12. A recombinant virus comprising the nucleic acid construct of claim 10.

13. The recombinant virus of claim 12, wherein the recombinant virus is an adeno-associated viral vector, an adenoviral vector, or a lentiviral vector.

14. A pharmaceutical composition comprising the nucleic acid construct of claim 10 and a pharmaceutically acceptable carrier.

15. A host cell comprising the nucleic acid construct of claim 10.

16. The host cell of claim 15, wherein the host cell is a human cell.

17. The host cell of claim 15, wherein the host cell is a neuronal cell or a pluripotent stem cell, wherein the stem cell is optionally an embryonic stem cell or an inducible pluripotent stem cell (iPSC).

18. A method of inhibiting expression of Nav1.7 in a human neuron, comprising introducing into the neuron the fusion protein of claim 1 thereby inhibiting the expression of Nav1.7 in the neuron.

19. The method of claim 18, wherein the human neuron is a nociceptive neuron in the dorsal root ganglia (DRG).

20. The method of claim 18, comprising introducing into the cell a recombinant adeno-associated virus (AAV) that expresses the fusion protein.

21. The method of claim 18, wherein the human neuron is in the body of a human patient.

22. A method of treating a pain disorder in a patient in need thereof, comprising administering to the patient a recombinant AAV or a nucleic acid construct encoding a fusion protein of claim 1.

23. The method of claim 22, wherein the recombinant AAV or nucleic acid construct is introduced to the patient via an intravenous, intrathecal, intracerebral, intracerebroventricular, intra-cisternal magna, epidural, or intranasal route.

24. The method of claim 21, wherein the patient is suffering from or at risk of developing a peripheral neuropathic pain disorder, optionally selected from
inherited erythromelalgia;
paroxysmal extreme pain disorder;
small fiber neuropathy, further optionally idiopathic small fiber neuropathy, small fiber neuropathy plus prediabetes, or diabetic small fiber neuropathy;
large fiber neuropathy;
trigeminal neuralgia;
post-herpetic neuralgia; and
painful diabetic neuropathy.

25. The method of claim 20, wherein the recombinant AAV is AAV9 or a pseudotype AAV derived from AAV9.

26. A method of inhibiting expression of Nav1.7 in a cell, preferably a human cell, comprising targeting a target site/sequence within or near a SCN9A gene with a zinc finger, TALE, or CRISPR repressor, thereby inhibiting expression of the gene.

27. (canceled)

28. (canceled)

29. The method of claim 26, wherein the target sequence: i) comprises at least 8 contiguous bps of any of SEQ ID NOs: 16-24; ii) comprises at least 8 non-contiguous bps of any of SEQ ID NOs: 16-24; or iii) comprises any one of SEQ ID NOs: 16-24.

30. (canceled)

31. (canceled)
